(19) United States
(12) Patent Application Publication
(10) Pub. No.: US 2021/0371461 A1
(43) Pub. Date: Dec. 2, 2021

(54) Novel chemiluminescent substrates for Factor Xa

(71) Applicant: Enzyre B.V., Nijmegen (NL)

(72) Inventors: Waander Laurens van Heerde, Nijmegen (NL); Mark van Geffen, Nijmegen (NL); Danique Steeghs, Nijmegen (NL)

(73) Assignee: Enzyre B.V., Nijmegen (NL)

(21) Appl. No.: 17/285,098

(22) PCT Filed: Oct. 17, 2019
(86) PCT No.: PCT/EP2019/078224

§ 371 Date: Apr. 14, 2021

(30) Foreign Application Priority Data
Oct. 17, 2018 (EP) 18201051.2

(51) Int. Cl. C07K 5/103; G01N 21/64; C12Q 1/66

(52) U.S. Cl. CPC C07K 5/101; G01N 2333/96463; C12Q 1/66; G01N 21/6408

(57) ABSTRACT

The present invention relates to chemiluminescent substrates for blood clotting enzyme Factor Xa. The substrates are particularly useful for assaying coagulation factors and for quantifying an anticoagulant in a sample.

### FIELD OF THE INVENTION

The present invention relates to chemiluminescent substrate molecules suitable for monitoring blood clotting enzyme Factor Xa. The substrates are particularly useful for assaying coagulation factors and for quantifying an anticoagulant in a sample.

### BACKGROUND ART

Coagulation of blood, also known as clotting, is the transformation of blood from a liquid to a gel, resulting in a blood clot. Coagulation is part of the hemostasis process and ultimately prevents excessive blood loss. Coagulation begins very soon after endothelium lining of a vessel is compromised. Exposure of the subendothelial space leads to the binding of plasma Factor VII (FVII) to tissue factor, which ultimately leads to fibrin formation. In so-called primary hemostasis, platelets immediately form a plug at the site of injury. So-called secondary hemostasis occurs simultaneously: coagulation factors respond in a complex cascade to form fibrin strands, which strengthens the platelet plug. Coagulation is highly conserved throughout biology.

FactorX (FX) is a key protein in coagulation. FX is the zymogen of activated FactorX (FXa). FX can be activated by the complex of Tissue Factor (TF) with Factor VII(a) (FVII(a)), or by the tenase complex. The tenase complex comprises activated Factor IX (FIXa) as the enzyme, activated FVIII (FVIIIa) as the cofactor, and the zymogen FX all bound to a phospholipid surface containing a certain percentage of negatively charged phospholipids. The complex converts FX into FXa.

Detection and quantification of FX or FXa activity is of interest for various reasons. FXa acts by cleaving prothrombin, yielding active thrombin; FX is the zymogen of FXa, so FX can be converted to FXa. Their detection therefore provides insight in blood clotting dynamics. FXa activity can also be used as a measure of anticoagulant activity: detection of FXa or of its formation can be applied to measure the concentration of anticoagulant proteins, like protein C and Protein S. Finally, FXa is also used as a read out of several anticoagulant drugs, like heparinoids or Direct Oral AntiCoagulant (DOAC) compounds against FXa.

Methods for quantifying blood clotting factors are well known (see S. S. van Berkel!, PhD thesis chapter 1, 2008, Radboud University Nijmegen). Methods for global hemostasis assays, a newer class of assays, are described by M. van Geffen (PhD Thesis chapters 2 and 3, 2012, Radboud University Nijmegen). Traditionally, in these known methods for quantification of blood clotting factors, fibrin formation is measured as a read out. Alternatively, later-developed chromogenic probes based on paranitroaniline conjugated to a peptide release free paranitroaniline (pNA) when the peptide is cleaved by an enzyme such as thrombin or FXa. The resulting color can be quantified and is a measure of enzyme activity. Examples are the commercially available S-2765 from Chromogenix, which is Z-D-Arg-Gly-Arg-pNA.HCl, and the commercially available S-2222 from Chromogenix, which comprises Bz-Ile-Glu-Gly-Arg-pNA.HCl IEGR is SEQ ID NO: 1).

Chromogenic tests have a disadvantage: because their methods depend upon the measurement of optical density, they cannot be carried out in a mixture that would become turbid due to clot formation, and the chromogenic yellow color interferes with the intrinsic yellow color of plasma. Therefore they should be carried out in defibrinated and consequently platelet poor plasma. Also they require subsampling because they cannot be measured in a continuous setting. Going from platelet poor plasma (PPP) to platelet rich plasma (PRP) to whole blood, the physiological system becomes more representative of what happens in the body though concomitantly, technically more difficult to assess.

Application of fluorogenic substrates made measurement in non defibrinated, platelet rich plasma and whole blood possible, and thus brought the assay system one step nearer to the physiological system while allowing continuous monitoring. The Van Berkel thesis cited above discusses the development of fluorogenic probes for thrombin.

The fluorometric probe ab204711 is commercially available (from Abcam PLC, UK). This Factor Xa Activity Assay Kit (Fluorometric) (ab204711) utilizes the ability of Factor Xa to cleave a synthetic substrate thereby releasing a fluorophore which can be quantified by fluorescence readers. A similar kit is available from Merck (catalogue number MAK238-1KT).

Other commercially available substrates are the SensoLyte® Rh110 Factor Xa Assay Kit from Eurogentec, which also uses a fluorogenic substrate that generates a fluorophore that can be detected after FXa cleavage of the substrate. The SensoLyte® 520 Factor Xa Assay Kit uses a 5-FAM/QXL™ 520 fluorescence resonance energy transfer (FRET) peptide, wherein fluorescence of 5-FAM is quenched by QXL™ 520. When FXa cleaves the intact peptide into two separate fragments, fluorescence of 5-FAM is recovered. This FRET peptide shows less interference from autofluorescence of test compounds and cellular components. WO02006/072602 describes the use of multiple fluorogenic substrates with different characteristics to allow the detection of several products in one sample.

Fluorogenic substrates have disadvantages: commercial platforms for analysis of the coagulation system generally do not support fluorometric analysis, thus requiring additional instrumentation. In addition, there is a desire to implement all coagulation tests wherever possible on one analyzer to simplify testing and minimize labor. The use of a separate instrument for measuring global assays thus reduces its applicability as a routine method. Fluorescent signals also have the drawback of not being linear with product concentration due to inner-filter effects and quenching effects.

Luminescent substrates do not have these disadvantages. They are more sensitive than chromogenic or fluorogenic substrates and do not require complex filters or excitation sources. U.S. Pat. No. 5,035,999 relates to luminescent substrates, but these substrates are not suitable for measurement in watery solutions (such as plasma), and neither for continuous measurement. WO2012096566 relates to substrates for thrombin or plasmin. Cosby et al. (“Custom enzyme substrates for luciferase-based assays”, Cell Notes, Issue 18 pages 9-11, 2007) relates to luminescent substrates, but these are not suitable for measurement in watery solutions (such as plasma) and neither for continuous measurement. Poor solubility often requires organic co-solvents that detract from the physiological conditions of an assay, or it requires larger volumes of sample or the addition of larger volumes of reagents.

It would be desirable for many clinical cases (e.g. in pediatric blood withdrawal, point of care monitoring in the home situation, in outbound critical care situations using an ambulance of helicopter) to have diagnostic tests for clotting factors that require a smaller reaction volume, thus requiring less blood. The system should be rather simple in its design and not require sophisticated technology, making it applicable in a (disposable) all in one point of care device. Moreover, a probe should allow a wide dynamic range, preferably over the three orders of magnitude offered by existing assays. The probe should be specific for its enzyme, such as FXa, and sensitive to allow its use in small sample volumes. The enabling of real-time measurement would improve flexibility of the assays in which these new probes could be used.

A need exists for a new assay for measuring FXa generation and/or measurement of other blood clotting factors or their activity, which does not have the above indicated drawbacks, that is it should be simpler and it should be able to measure the generation of blood clotting and fibrinolytic factors in a direct manner, preferably in a linear mode. It is an object of the present invention to provide substrates and methods for such an assay.

### SUMMARY OF THE INVENTION

The invention is in the field of new molecules as substrates for use in assays related to medicine, in particular in the field of blood coagulation. The molecules release a chemiluminescent substance when they are cleaved by the appropriate enzyme. More specifically, the new molecules can be used in a novel method for directly measuring the activity of hemostasis factors. To be more precise, the invention is related to molecules for use in a method to quantify coagulation factors, or for use in a method to analyze FXa generation in a global assay. The coagulation factors to be quantified are at least the procoagulant factors FIX, FVIII, FVII and the anticoagulant factor antithrombin, protein C, and protein S. The invention is also related to a method to quantify heparinoids and Direct Oral AntiCoagulants (DOACs) against Xa.

In one aspect the invention relates to a compound of general formula (I-3) or (I-4),

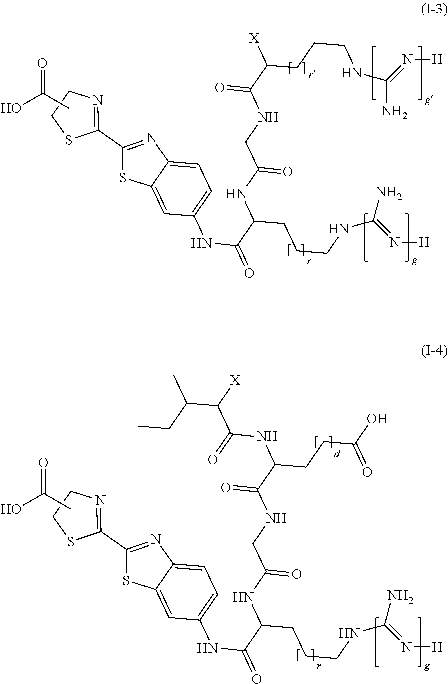

wherein r is 0, 1, 2, or 3; r′ is 0, 1, 2, or 3; d is 0, 1, or 2; g is 0 or 1; g′ is 0 or 1; and X is a terminal moiety selected from NH₂, OH, O(C₁₋₆alkyl), (OCH₂CH₂)₁₋₆OH, (OCH₂CH₂)₁₋₆O(C₁₋₆alkyl), NHC(═O)(O)₀₋₁(C₁₋₆alkyl), NHC(═O)(O)₀₋₁(C₁₋₆alkylene)(OCH₂CH₂)₁₋₆OH, NHC(═O)(O)₀₋₁(C₁₋₆alkylene)(OCH₂CH₂)₁₋₆O(C₁₋₆alkyl), NHC(═O)(O)₀₋₁(C₁₋₆alkylene)O(C₁₋₆alkyl), NHC(═O)(O)₀₋₁(C₁₋₆alkylene)OH, and NP′ wherein P′ is an amine protecting group; optionally wherein the aminoluciferin moiety is replaced by a different chemiluminescent amine; or a physiologically acceptable salt thereof.

In another aspect the invention relates to a combination comprising a compound as defined in the first aspect and at least one further compound selected from the group consisting of luciferase, ATP, an Mg²⁺ source, and a coagulation factor such as factor Xa. In preferred embodiments the combination relates to a device for measuring chemiluminescence, the device comprising a compound of the first aspect.

In another aspect the invention relates to a method for quantifying a coagulation factor in a sample, the method comprising the steps of a) contacting the sample with a composition comprising a compound of the first aspect to release aminoluciferin; b) contacting the aminoluciferin with luciferase; and c) determining the relative light intensity generated by the luciferase. This method can be used to quantify an anticoagulant in a sample when FXa is provided in step a).

### DESCRIPTION OF EMBODIMENTS

The inventors have surprisingly found that a class of chemiluminescent compounds can be used in hemostasis assays to detect FXa activity, or to indirectly detect the activity of other factors via FXa detection. Accordingly, in a first aspect the invention provides a compound of general formula (I-3) or (I-4),

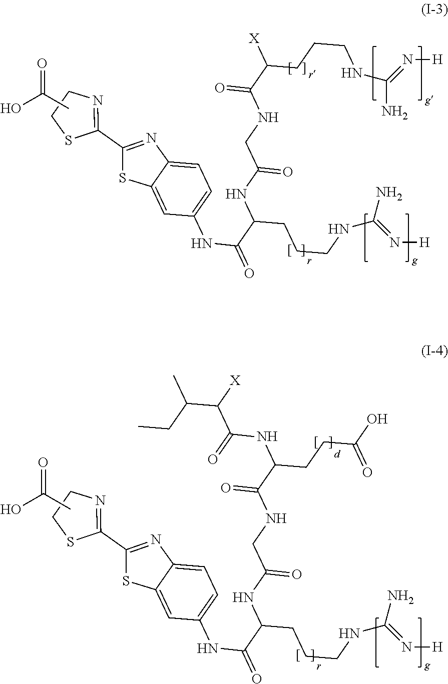

wherein

r is 0, 1, 2, or 3;

r′ is 0, 1, 2, or 3;

d is 0, 1, or 2;

g is 0 or 1;

g′ is 0 or 1; and

X is a terminal moiety selected from NH₂, OH, O(C₁₋₆alkyl), (OCH₂CH₂)₁₋₆OH,

(OCH₂CH₂)₁₋₆O(C₁₋₆alkyl), NHC(═O)(O)₀₋₁(C₁₋₆alkyl), NHC(═O)(O)₀₋₁(C₁₋₆alkylene)(OCH₂CH₂)₁₋₆OH, NHC(═O)(O)₀₋₁(C₁₋₆alkylene)(OCH₂CH₂)₁₋₆O(C₁₋₆alkyl), NHC(═O)(O)₀₋₁(C₁₋₆alkylene)O(C₁₋₆alkyl), NHC(═O)(O)₀₋₁(C₁₋₆alkylene)OH, and NP′ wherein P′ is an amine protecting group; optionally wherein the aminoluciferin moiety is replaced by a different chemiluminescent amine; optionally wherein a carboxylic acid moiety is esterified with a C₁₋₄alkanol; or a physiologically acceptable salt thereof. Such a compound is referred to hereinafter as a compound according to the invention

Compounds of general formula I-3 comprise a tripeptide linked to a chemiluminescent amine via an amide bond. Compounds of general formula I-4 comprise a tetrapeptide linked to a chemiluminescent amine via an amide bond. In cases where X forms an amino acid, the term tripeptide is still used for general formula I-3, and tetrapeptide for general formula I-4; While a tetrapeptide comprises a tripeptide, context will make clear when these terms are intended to refer to the structures of general formula 1-3 or 1-4, instead of referring to tripeptides and tetrapeptides in general. General formula 1-3 and 1-4 share many characteristics. Accordingly, as used herein, reference to a general formula without an indication such as -3 or -4 is intended to relate to both -3 and -4 variants of that general formula. For example, general formula I relates to both I-3 and I-4, and general formula Is relates to both I3s and I-4s. For ease of reference, amino acids present in the tripeptide or tetrapeptide are numbered starting at the cationic amino acid directly adjacent to the chemiluminescent amine. Accordingly, the residue linked to X in general formula I-3 is residue 3, whereas it is residue 4 in general formula I-4.

Chemiluminescent molecules are known in the art. Examples of chemiluminescent amines are amines of luciferin such as amines of firefly luciferin, latia luciferin, bacterial luciferin, coelenterazine, cypridinluciferin, or 3-hydroxy hyspidin. The chemiluminescent amine is preferably aminoluciferin of firefly luciferin or optionally a C₁₋₄aalkyl ester thereof such as 2-(6-amino-1,3-benzothiazol-2-yl)-4,5-dihydrothiazole-(4/5)-carboxylic acid, more preferably a 2-(6-amino-1,3-benzothiazol-2-yl)-4,5-dihydrothiazole-4-carboxylic acid or optionally a C₁₋₄aalkyl ester thereof such as (4S)-2-(6-amino-1,3-benzothiazol-2-yl)-4,5-dihydrothiazole-4-carboxylic acid or optional C₁₋₄alkyl esters thereof, most preferably (4S)-2-(6-amino-1,3-benzothiazol-2-yl)-4,5-dihydrothiazole-4-carboxylic acid.

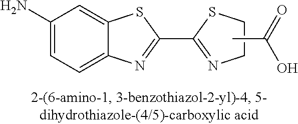

In preferred embodiments is provided the compound according to the invention, wherein the compound is of general formula (II-3) or (II-4):

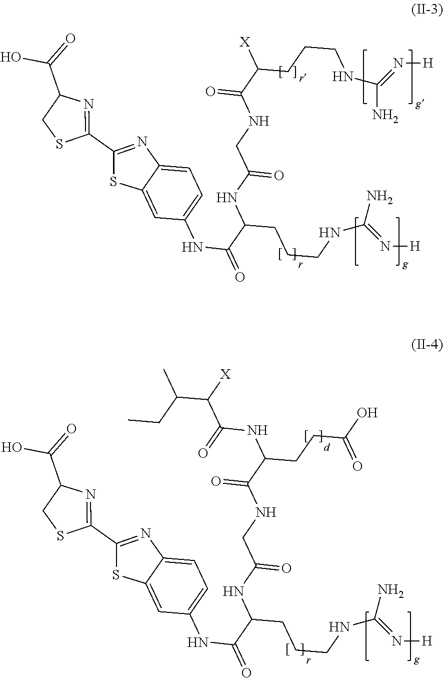

wherein r, r′, d, g, g′, and X are as defined elsewhere herein, preferably wherein the 4-carboxylic acid of the aminoluciferin moiety is S.

The chemiluminescent amines cannot be substrates to their corresponding luciferase enzymes when they are comprised in compounds according to the invention. Cleavage by FXa liberates the chemiluminescent amine and enables conversion by the corresponding enzyme, leading to emission of a photon, or in other words to generation of a light quant.

Compounds according to the invention can have carboxylic acid moieties. Optionally, these can be esterified with a C₁₋₄alkanol. Examples of C₁₋₄alkanols are methanol, ethanol, n-propanol, isopropanol, tent-butanol, n-butanol, butan-2-ol, and isobutanol. In this context, a preferred C₁₋₄alkanol is methanol. The C₁₋₄aalkanol is optionally substituted with 1, 2, 3, 4, 5, 6, 7, 8, or 9 halogen atoms such as fluorine, and is optionally substituted with methoxy or ethoxy. It can also be optionally unsaturated, such as vinyl alcohol or allyl alcohol. Compounds according to the invention can be mixtures of such esters and the free acid, for example 1:1 mixtures of the methyl ester at position 3 of general formula I-4 or I-4.

Compounds according to the invention can thus also be represented by general formula 0:

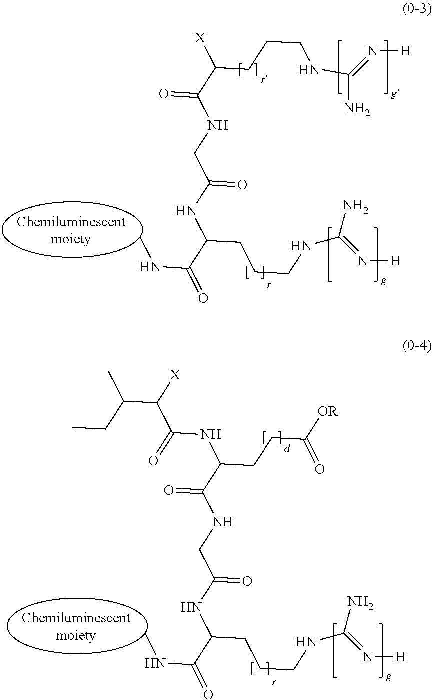

wherein r, r′ d, g, g′, and X are as defined above, and wherein R is H or C₁₋₄aalkyl, preferably R is H. C₁₋₄aalkyl is optionally substituted with 1, 2, 3, 4, 5, 6, 7, 8, or 9 halogen atoms such as fluorine, and is optionally substituted with methoxy or ethoxy. It can also be optionally unsaturated, such as vinyl or allyl.

Compounds according to the invention can be physiologically acceptable salts. Such salts are known in the art, and a skilled person can select a suitable salt form. In this context a physiologically acceptable salt is a salt that can still be used as a substrate in assays as exemplified later herein. Examples of physiologically acceptable salts are acid addition salts or alkali salts such as sodium salts or potassium salts. Acid addition salts are preferred. Suitable acid addition salts are salts formed through addition of formic acid, acetic acid, propionic acid, trifluoroacetic acid, mesylic acid, tosylic acid, or hydrohalic acids such as HBr or HCI. In preferred embodiments is provided the compound according to the invention, wherein the compound is an acid addition salt optionally selected from a HCI salt, an acetic acid salt, a formic acid salt, a TFA salt, and a mesylic acid salt, preferably a HCI salt or a TFA salt, most preferably a TFA salt.

The third residue of a compound of general formula I-4 has a side chain that can be 1, 2, or 3 methylene units long. This is because d is 0, 1, or 2. The third residue can be aspartic acid when d is 0; the third residue can be glutamic acid when d is 1. Such compounds have shown good results. Accordingly, in preferred embodiments is provided the compound according to the invention, wherein d is 0 or 1, preferably 1.

The first residue of general formula I has a cationic side chain, and so does the third residue of general formula I-3. These cationic side chains can be based on a primary amine when g or g′ is 0, and can be based on a guanidine moiety when g or g′ is 1. Substrates wherein g is 1 have a good affinity for FXa; accordingly, in preferred embodiments, g is 1. In preferred embodiments, g′ is 1. In more preferred embodiments, g and g′ are 1. When g is 1, it is preferred that r is 1, as arginine has r is 1 and g is 1. Similarly, when g is 0, it is preferred that r is 2, as lysine has r is 2 and g is 0. Similarly, when g′ is 1, it is preferred that r′ is 1. Similarly, when g′ is 0, it is preferred that r′ is 2. In preferred embodiments is provided the compound according to the invention, wherein r is 1 or 2, or wherein g is 1, preferably wherein r is 1, more preferably wherein r is 1 and g is 1. Arginine and lysine are preferred for quantifying serine protease activity including FXa.

Arginine can be preferred when a more fast-acting substrate is desired. Lysine can be preferred when a more slow-acting substrate is desired. The affinity of a compound according to the invention for FXa is of influence on assay parameters. For global assays, a substrate with lower affinity is desirable. For quantitative assays, a substrate with a higher affinity is desirable.

Substrates with a particular chirality were found to have an improved affinity for FXa. Preferably, the first residue is L. There is no chiral preference for the second residue, which lacks a side chain so it can be said to be glycine. There is no general preference for the third residue, but for a general formula with a tripeptide it is preferably D, while for a general formula with a tetrapeptide it is preferably L. The fourth residue is preferably L. Accordingly, in preferred embodiments a compound according to the invention has a first residue that is L. In more preferred embodiments a compound according to the invention has a first residue that is L and a third residue that is D when the compound is of general formula I-3, or a third and a fourth residue that is L when the compound is of general formula I-4. Accordingly, in preferred embodiments is provided the compound according to the invention, wherein the compound is of general formula (I 3s) or (I-4s),

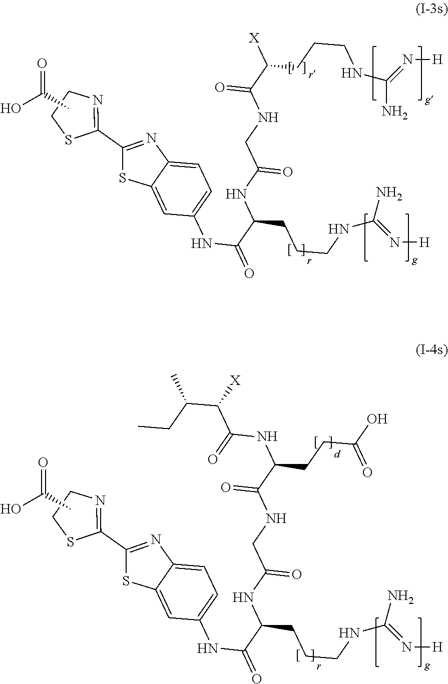

wherein r, r′, d, g, g′, and X are as defined elsewhere herein.

X is a terminal moiety selected from NH₂, OH, O(C₁₋₆alkyl), (OCH₂CH₂)₁₋₆OH, (OCH₂CH₂)₁₋₆O(C₁₋₆alkyl), NHC(═O)(O)₀₋₁(C₁₋₆alkyl), NHC(═O)(O)₀₋₁(C₁₋₆alkylene)(OCH₂CH₂)₁₋₆OH, NHC(═O)(O)₀₋₁(C₁₋₆alkylene)(OCH₂CH₂)₁₋₆O(C_{1- 6}alkyl), NHC(═O)(O)₀₋₁(C₁₋₆alkylene)O(C₁₋₆alkyl), NHC(═O)(O)₀₋₁(C₁₋₆alkylene)OH, and NP′ wherein P′ is an amine protecting group. In this context, a terminal moiety is not necessarily at the terminus of the compound according to the invention, but it is at the terminus (generally the N-terminal side) of the tripeptide or tetrapeptide of general formula I. It was found that terminal moieties comprising (OCH₂CH₂)1-6 moieties led to compounds according to the invention with good aqueous solubility. Accordingly, preferably X is (OCH₂CH₂)₁₋₆OH, (OCH₂CH₂)₁₋₆O(C₁₋₆alkyl), NHC(═O)(₀₋₁(C₁₋₆alkylene)(OCH₂CH₂)₁₋₆OH, or NHC(═O)(O)₀₋₁(C₁₋₆alkylene)(OCH₂CH₂)₁₋₆O(C_{1- 6}alkyl).

When X is NHC(═O)(O)₀₋₁(C₁₋₆alkyl), NHC(═O)(O)₀₋₁(C₁₋₆alkylene)(OCH₂CH₂)₁₋₆OH, NHC(═O)(O)₀₋₁(C₁₋₆alkylene)(OCH₂CH₂)₁₋₆O(C_{1- 6}alkyl), NHC(═O)(O)₀₋₁(C₁₋₆alkylene)O(C₁₋₆alkyl), or NHC(═O)(O)₀₋₁(C₁₋₆alkylene)OH, X is linked to the compound according to the invention via an amide or via a carbamate, owing to the NHC(═O)(O)₀₋₁ motif. Preferably, X is an amide in these cases. Accordingly, in preferred embodiments, X is NHC(═O)(C₁₋₆alkyl), NHC(═O)(O)₀₋₁(C₁₋₆alkylene)(OCH₂CH₂)₁₋₆O(C_{1- 6}alkyl), NHC(═O)(O)₀₋₁(C₁₋₆alkylene)O(C₁₋₆alkyl), NHC(═O)(O₁₋₆alkylene)O(C₁₋₆alkyl), or NHC(═O)(C₁₋₆alkylene)OH, more preferably X is NHC(═O)(C₁₋₆alkylene)(OCH₂CH₂)₁₋₆OH, or NHC(═O)(C₁₋₆alkylene)(OCH₂CH₂)₁₋₆O(C₁₋₆alkyl).

As comprised in X, C₁₋₆alkyl can be methyl, ethyl, n-propyl, isopropyl, n-butyl, sec-butyl, tert-butyl, isobutyl, n-pentyl, tert-pentyl, neopentyl, isopentyl, sec-pentyl, 3-pentyl, sec-isopentyl, 2-methylbutyl, n-hexyl, or isohexyl, preferably C₁₋₆alkyl is methyl, ethyl, n-propyl, isopropyl, n-butyl, sec-butyl, tert-butyl, or isobutyl, more preferably it is methyl, ethyl, isopropyl, or tert-butyl, most preferably methyl.

As comprised in X, C₁₋₆alkylene can be methylene, methylmethylene, ethylene, n-propylene, methylethylene, n-butylene, 1-methylpropylene, 1,1-dimethylethylene, n-pentylene, isopentylene, 2-methylbutylene, n-hexylene, or isohexylene, preferably C₁₋₆alkylene is methylene, ethylene, n-propylene, n-butylene, n-pentylene, or n-hexylene, more preferably it is methylene, ethylene, n-propylene, n-butylene, or n-pentylene, most preferably methylene.

As comprised in X, (OCH₂CH₂)₁₋₆ represents ethylene glycol repeats. Preferably, (OCH₂CH₂)₁₋₆ is (OCH₂CH₂)₂, (OCH₂CH₂)₃, (OCH₂CH₂)₄, or (OCH₂CH₂)₅, more preferably (OCH₂CH₂)₂, (OCH₂CH₂)₃, or (OCH₂CH₂)₄, even more preferably (OCH₂CH₂)₂ or (OCH₂CH₂)₃, most preferably (OCH₂CH₂)₂. When X comprises (OCH₂CH₂)₁-₆, any C₁₋₆alkylene is preferably methylene or ethylene, more preferably methylene, and any C₁₋₆alkyl is preferably methyl or tert-butyl, more preferably methyl.

X can also be NP′. P′ is an amine protective group. Amine protective groups are known in the art and can protect nitrogen atoms in amines as well as other nitrogen atoms. Examples of suitable amine protective groups are extensively described in the art, e.g. by P. G. M. Wuts and T. W. Greene in Greene's Protective Groups in Organic Synthesis, Fourth Edition, 2006 (ISBN: 978-0-471-69754-1). The person skilled in the art will be able to select suitable protective groups to be used in accordance with the present invention. Examples of suitable amine protective groups are trityl, allyl, benzyl (Bn), 9-fluorenylmethyl oxycarbonyl (Fmoc), t-butyloxycarbonyl (Boc), benzyloxycarbonyl (Z), 2-trimethylsilylethyloxycarbonyl, tosyl (Ts), acetyl (Ac), trifluoroacetyl, phthalimide, benzylideneamine, and allyloxycarbonyl (Alloc). Preferred groups for P′ are trityl, allyl, benzyl (Bn), 9-fluorenylmethyl oxycarbonyl (Fmoc), t-butyloxycarbonyl (Boc), benzyloxycarbonyl (Z), tosyl (Ts), and allyloxycarbonyl (Alloc). More preferred groups for P′ are allyloxycarbonyl and t-butyloxycarbonyl. P′ is always attached to nitrogen, and multiple instances of an amine protective group can protect a single amine or nitrogen atom—as understood by a skilled person, amine protecting groups can also protect nitrogen atoms that are not strictly an amine, such as nitrogen atoms in pyridine rings. Hydrogen should be added or omitted to maintain correct valency for nitrogen to which P′ is attached. Accordingly valency permitting, NP' can be interchanged with NHP', and NHP' can be interchanged with NP'. In preferred embodiments is provided the compound according to the invention, wherein P′ is selected from the group consisting of trityl, allyl, benzyl (Bn), benzoyl (Bz), 9-fluorenylmethyl oxycarbonyl (Fmoc), t-butyloxycarbonyl (Boc), benzyloxycarbonyl (Z), 2-trimethylsilylethyloxycarbonyl, tosyl (Ts), acetyl (Ac), trifluoroacetyl, phthalimide, benzylideneamine, and allyloxycarbonyl (Alloc), preferably from the group consisting of benzyl (Bn), 9-fluorenylmethyl oxycarbonyl (Fmoc), t-butyloxycarbonyl (Boc), benzyloxycarbonyl (Z), tosyl (Ts), and allyloxycarbonyl (Alloc), more preferably P′ is benzyloxycarbonyl.

In preferred embodiments, X is represented by general formula X-2:

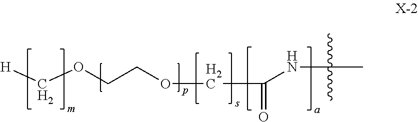

wherein m is 0, 1, 2, 3, 4, 5, or 6; p is 0, 1, 2, 3, 4, 5, or 6; s is 0, 1, 2, 3, 4, 5, or 6; and a is 0 or 1; wherein methylene moieties comprised in m, s, or p can be substituted by methyl, ethyl, or isopropyl, optionally wherein the resulting alkyl or alkylene moiety can be substituted with one or more halogen atom such as fluorine or chlorine. Accordingly, in preferred embodiments is provided the compound according to the invention, wherein the compound is of general formula (III-3) or (III-4),

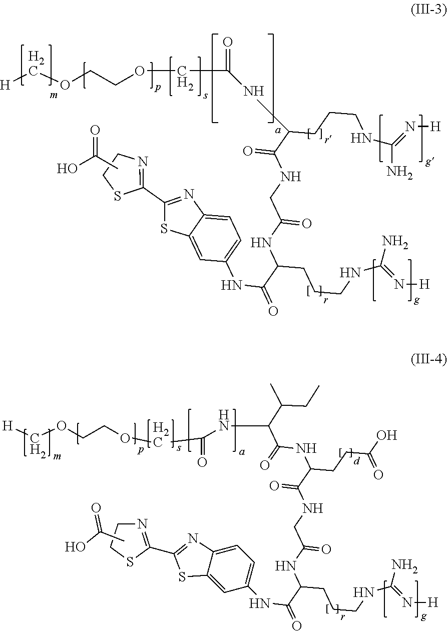

Wherein

d is 0, 1, or 2;

r is 0, 1, 2, or 3;

r′ is 0, 1, 2, or 3;

g is 0 or 1;

g′ is 0 or 1;

m is 0, 1, 2, 3, 4, 5, or 6;

s 0, 1, 2, 3, 4, 5, or 6;

s is 0, 1, 2, 3, 4, 5, or 6; and

a is 0 or 1;

wherein methylene moieties comprised in m, s, or p can be substituted by methyl, ethyl, or isopropyl, optionally wherein the resulting alkyl moiety can be substituted with one or more halogen atom such as fluorine or chlorine. Hydrogen atoms can be added or removed to preserve correct valency. Preferably, when methylene moieties comprised in m are substituted, m is 1 or 2; more preferably, when methylene moieties comprised in m are substituted m is 2 and the methylene moiety adjacent to the oxygen atom is substituted with one or two methyl moieties. This forms isopropoxy and tert-butoxy, respectively. When p is 1, 2, 3, 4, 5, or 6, it is preferred that m is 0, 1, or 2, and that when m is 2, the methylene moiety adjacent to the oxygen atom is substituted with one or two methyl moieties.

a is an integer that is 0 or 1, and accordingly it can introduce an amide moiety in X. Owing to their convenient synthetic accessibility, compounds wherein a is 1 are preferred.

s is an integer that is 0, 1, 2, 3, 4, 5, 6, or 6. When s is 0 and a is 1, X comprises a carbamate moiety. In other instances s can form a linker moiety that connects the peptide with an oligoethylene-glycol moiety when present, or with a terminal alcohol or methyl ether. Methylene moieties in s can be optionally substituted with methyl, ethyl, or methoxy, and are optionally substituted with one or more halogen atom such as fluorine or chlorine. When p is 1, 2, 3, 4, 5, or 6, it is preferred that s is 0, 1, or 2, more preferably 1 or 2, more preferably 1. When p is 1, 2, 3, 4, 5, or 6, it is preferred that a is 0 and s is 0, 1, or 2, more preferably that a is 1 and s is 1 or 2, more preferably 1.

p is an integer that is 0, 1, 2, 3, 4, 5, 6, or 6. It can form an oligoethylene glycol moiety that can have a positive effect on aqueous solubility of the compounds of the invention. Ethylene glycol moieties in p can be optionally substituted with methyl, and are optionally substituted with one or more halogen atom such as fluorine or chlorine. p is preferably 1, 2, 3, or 4, more preferably 1, 2, or 3, even more preferably 2 or 3, most preferably 2.

In preferred embodiments the invention provides the compound of general formula III-3 or III-4,

wherein

d is 0 or 1, preferably 1; and/or

r is 1 or 2, preferably 1; and/or

r′ is 1 or 2, preferably 1; and/or

g is 0 or 1, preferably 1; and/or

g′ is 0 or 1, preferably 1; and/or

m is 0 or 1; and/or

p is 1, 2, or 3, preferably 2; and/or

s is 1 or 2, preferably 1; and/or

a is 1.

Table 1 shows preferred embodiments of general formula I-4, I-4s, II-4, or III-4.

TABLE 1

| Embodiment | r | g | d |
|---|---|---|---|
| AA | 0 | 0 | 0 |
| AB | 1 | 0 | 0 |
| AC | 2 | 0 | 0 |
| AD | 3 | 0 | 0 |
| AE | 0 | 1 | 0 |
| AF | 1 | 1 | 0 |
| AG | 2 | 1 | 0 |
| AH | 3 | 1 | 0 |
| AI | 0 | 0 | 1 |
| AJ | 1 | 0 | 1 |
| AK | 2 | 0 | 1 |
| AL | 3 | 0 | 1 |
| AM | 0 | 1 | 1 |
| AN | 1 | 1 | 1 |
| AO | 2 | 1 | 1 |
| AP | 3 | 1 | 1 |
| AQ | 0 | 0 | 2 |
| AR | 1 | 0 | 2 |
| AS | 2 | 0 | 2 |
| AT | 3 | 0 | 2 |
| AU | 0 | 1 | 2 |
| AV | 1 | 1 | 2 |
| AW | 2 | 1 | 2 |
| AX | 3 | 1 | 2 |

In preferred embodiments, compounds of general formula I-4, I-4s, II-4, or III-4 are selected from AE, AF, AG, AH, AM, AN, AO, AP, AU, AV, AW, and AX. In preferred embodiments, compounds of general formula I-4, I-4s, II-4, or III-4 are selected from AE, AF, AG, AH, AM, AN, AO, and AP. In preferred embodiments, compounds of general formula I-4, I-4s, II-4, or III-4 are selected from AE, AF, AG, AM, AN, and AO, more preferably selected from AF, AG, AN and AO, even more preferably selected from AF and AN, most preferably it is AN.

Table 2 shows preferred embodiments of general formula I-3, I-3s, II-3, or III-3.

TABLE 2

| Embodiment | r | g | r′ | g′ |
|---|---|---|---|---|
| BA | 0 | 0 | 0 | 0 |
| BB | 1 | 0 | 0 | 0 |
| BC | 2 | 0 | 0 | 0 |
| BD | 3 | 0 | 0 | 0 |
| BE | 0 | 1 | 0 | 0 |
| BF | 1 | 1 | 0 | 0 |
| BG | 2 | 1 | 0 | 0 |
| BH | 3 | 1 | 0 | 0 |
| BI | 0 | 0 | 1 | 0 |
| BJ | 1 | 0 | 1 | 0 |
| BK | 2 | 0 | 1 | 0 |
| BL | 3 | 0 | 1 | 0 |
| BM | 0 | 1 | 1 | 0 |
| BN | 1 | 1 | 1 | 0 |
| BO | 2 | 1 | 1 | 0 |
| BP | 3 | 1 | 1 | 0 |
| BQ | 0 | 0 | 2 | 0 |
| BR | 1 | 0 | 2 | 0 |
| BS | 2 | 0 | 2 | 0 |
| BT | 3 | 0 | 2 | 0 |
| BU | 0 | 1 | 2 | 0 |
| BV | 1 | 1 | 2 | 0 |
| BW | 2 | 1 | 2 | 0 |
| BX | 3 | 1 | 2 | 0 |
| BY | 0 | 0 | 3 | 0 |
| BZ | 1 | 0 | 3 | 0 |
| BAA | 2 | 0 | 3 | 0 |
| BBA | 3 | 0 | 3 | 0 |
| BCA | 0 | 1 | 3 | 0 |
| BDA | 1 | 1 | 3 | 0 |
| BEA | 2 | 1 | 3 | 0 |
| BFA | 3 | 1 | 3 | 0 |
| BGA | 0 | 0 | 0 | 1 |
| BHA | 1 | 0 | 0 | 1 |
| BIA | 2 | 0 | 0 | 1 |
| BJA | 3 | 0 | 0 | 1 |
| BKA | 0 | 1 | 0 | 1 |
| BLA | 1 | 1 | 0 | 1 |
| BMA | 2 | 1 | 0 | 1 |
| BNA | 3 | 1 | 0 | 1 |
| BOA | 0 | 0 | 1 | 1 |
| BPA | 1 | 0 | 1 | 1 |
| BQA | 2 | 0 | 1 | 1 |
| BRA | 3 | 0 | 1 | 1 |
| BSA | 0 | 1 | 1 | 1 |
| BTA | 1 | 1 | 1 | 1 |
| BUA | 2 | 1 | 1 | 1 |
| BVA | 3 | 1 | 1 | 1 |
| BWA | 0 | 0 | 2 | 1 |
| BXA | 1 | 0 | 2 | 1 |
| BYA | 2 | 0 | 2 | 1 |
| BZA | 3 | 0 | 2 | 1 |
| BAB | 0 | 1 | 2 | 1 |
| BBB | 1 | 1 | 2 | 1 |
| BCB | 2 | 1 | 2 | 1 |
| BDB | 3 | 1 | 2 | 1 |
| BEB | 0 | 0 | 3 | 1 |
| BFB | 1 | 0 | 3 | 1 |
| BGB | 2 | 0 | 3 | 1 |
| BHB | 3 | 0 | 3 | 1 |
| BIB | 0 | 1 | 3 | 1 |
| BJB | 1 | 1 | 3 | 1 |
| BKB | 2 | 1 | 3 | 1 |
| BLB | 3 | 1 | 3 | 1 |

Table 3 shows preferred embodiments of X wherein it is of general formula X-2.

TABLE 3

| Embodiment | m | p | s | a |
|---|---|---|---|---|
| XAA | 0 | 1 | 0 | 0 |
| XAB | 0 | 2 | 0 | 0 |
| XAC | 0 | 3 | 0 | 0 |
| XAD | 0 | 4 | 0 | 0 |
| XAE | 1 or 2 | 1 | 0 | 0 |
| XAF | 1 or 2 | 2 | 0 | 0 |
| XAG | 1 or 2 | 3 | 0 | 0 |
| XAH | 1 or 2 | 4 | 0 | 0 |
| XAI | 0 | 1 | 1 | 0 |
| XAJ | 0 | 2 | 1 | 0 |
| XAK | 0 | 3 | 1 | 0 |
| XAL | 0 | 4 | 1 | 0 |
| XAM | 1 or 2 | 1 | 1 | 0 |
| XAN | 1 or 2 | 2 | 1 | 0 |
| XAO | 1 or 2 | 3 | 1 | 0 |
| XAP | 1 or 2 | 4 | 1 | 0 |
| XAQ | 0 | 1 | 2 | 0 |
| XAR | 0 | 2 | 2 | 0 |
| XAS | 0 | 3 | 2 | 0 |
| XAT | 0 | 4 | 2 | 0 |
| XAU | 1 or 2 | 1 | 2 | 0 |
| XAV | 1 or 2 | 2 | 2 | 0 |
| XAW | 1 or 2 | 3 | 2 | 0 |
| XAX | 1 or 2 | 4 | 2 | 0 |
| XAY | 0 | 1 | 3 | 0 |
| XAZ | 0 | 2 | 3 | 0 |
| XBA | 0 | 3 | 3 | 0 |
| XBB | 0 | 4 | 3 | 0 |
| XBC | 1 or 2 | 1 | 3 | 0 |
| XBD | 1 or 2 | 2 | 3 | 0 |
| XBE | 1 or 2 | 3 | 3 | 0 |
| XBF | 1 or 2 | 4 | 3 | 0 |
| XBG | 0 | 1 | 0 | 1 |
| XBH | 0 | 2 | 0 | 1 |
| XBI | 0 | 3 | 0 | 1 |
| XBJ | 0 | 4 | 0 | 1 |
| XBK | 1 or 2 | 1 | 0 | 1 |
| XBL | 1 or 2 | 2 | 0 | 1 |
| XBM | 1 or 2 | 3 | 0 | 1 |
| XBN | 1 or 2 | 4 | 0 | 1 |
| XBO | 0 | 1 | 1 | 1 |
| XBP | 0 | 2 | 1 | 1 |
| XBQ | 0 | 3 | 1 | 1 |
| XBR | 0 | 4 | 1 | 1 |
| XBS | 1 or 2 | 1 | 1 | 1 |
| XBT | 1 or 2 | 2 | 1 | 1 |
| XBU | 1 or 2 | 3 | 1 | 1 |
| XBV | 1 or 2 | 4 | 1 | 1 |
| XBW | 0 | 1 | 2 | 1 |
| XBX | 0 | 2 | 2 | 1 |
| XBY | 0 | 3 | 2 | 1 |
| XBZ | 0 | 4 | 2 | 1 |
| XCA | 1 or 2 | 1 | 2 | 1 |
| XCB | 1 or 2 | 2 | 2 | 1 |
| XCC | 1 or 2 | 3 | 2 | 1 |
| XCD | 1 or 2 | 4 | 2 | 1 |
| XCE | 0 | 1 | 3 | 1 |
| XCF | 0 | 2 | 3 | 1 |
| XCG | 0 | 3 | 3 | 1 |
| XCH | 0 | 4 | 3 | 1 |
| XCI | 1 or 2 | 1 | 3 | 1 |
| XCJ | 1 or 2 | 2 | 3 | 1 |
| XCK | 1 or 2 | 3 | 3 | 1 |
| XCL | 1 or 2 | 4 | 3 | 1 |

In preferred embodiments of embodiments of table 3 wherein m is 1 or 2, it is 1. Preferably, in embodiments of table 3 wherein m is 1 or 2, the methylene moiety adjacent to the oxygen atom is substituted with one or two methyl moieties when m is 2. In preferred embodiments, X is selected from the group consisting of XBO, XBP, XBQ, XBR, XBS, XBT, XBU, XBV, XBW, XBX, XBY, XBZ,

XCA, XCB, XCC, and XCD, more preferably from the group consisting of XBO, XBP, XBQ, XBR, XBS, XBT, XBU, and XBV, more preferably from the group consisting of XBO, XBP, XBQ, XBS, XBT, and XBU, most preferably XBT.

In preferred embodiments, compounds of general formula I-4, I-4s, II-4, or III-4 are selected from AE, AF, AG, AH, AM, AN, AO, AP, AU, AV, AW, and AX, most preferably AN; wherein X is selected from the group consisting of XBO, XBP, XBQ, XBR, XBS, XBT, XBU, XBV, XBW, XBX, XBY, XBZ, XCA, XCB, XCC, and XCD, more preferably from the group consisting of XBO, XBP, XBQ, XBR, XBS, XBT, XBU, and XBV, more preferably from the group consisting of XBO, XBP, XBQ, XBS, XBT, and XBU, most preferably XBT. In preferred embodiments, compounds of general formula I-4, I-4s, II-4, or III-4 are selected from AE, AF, AG, AH, AM, AN, AO, and AP, most preferably AN; wherein X is selected from the group consisting of XBO, XBP, XBQ, XBR, XBS, XBT, XBU, XBV, XBW, XBX, XBY, XBZ, XCA, XCB, XCC, and XCD, more preferably from the group consisting of XBO, XBP, XBQ, XBR, XBS, XBT, XBU, and XBV, more preferably from the group consisting of XBO, XBP, XBQ, XBS, XBT, and XBU, most preferably XBT. In preferred embodiments, compounds of general formula I-4, I-4s, II-4, or III-4 are selected from AE, AF, AG,

AM, AN, and AO, most preferably AN; wherein X is selected from the group consisting of XBO, XBP, XBQ, XBR, XBS, XBT, XBU, XBV, XBW, XBX, XBY, XBZ, XCA, XCB, XCC, and XCD, more preferably from the group consisting of XBO, XBP, XBQ, XBR, XBS, XBT, XBU, and XBV, more preferably from the group consisting of XBO, XBP, XBQ, XBS, XBT, and XBU, most preferably XBT.

Table 4 shows preferred embodiments of compounds according to the invention. More preferred compounds according to the invention are physiologically acceptable salts of the compounds shown in table 4, more preferably acid addition salts, most preferably TFA salts. In preferred embodiments is provided the compound according to the invention, wherein it is selected from MePEG2-IEGR, MePEG2-IDGR, MePEG3-IEGR, MePEG3-IDGR, MePEG2-RGR, MePEG2-RGK, MePEG3-RGR, and MePEG3-RGK.

TABLE 4

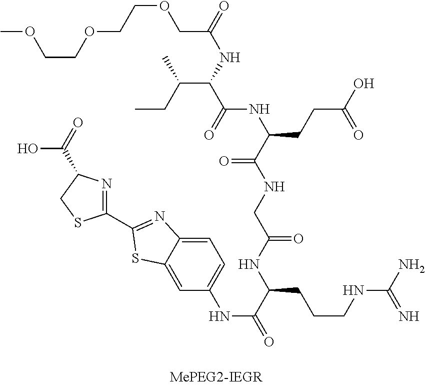 [SMILES: CC[C@H](C)[C@H](NC(=O)COCCOCCOC)C(=O)N[C@@H](CCC(=O)O)C(=O)NCC(=O)N[C@@H](CCCNC(=N)N)C(=O)Nc1ccc2nc(C3=N[C@@H](C(=O)O)CS3)sc2c1]

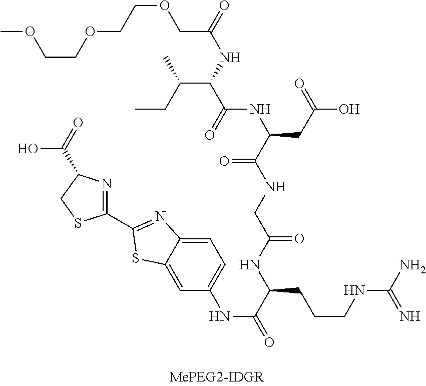 [SMILES: CC[C@H](C)[C@H](NC(=O)COCCOCCOC)C(=O)N[C@@H](CC(=O)O)C(=O)NCC(=O)N[C@@H](CCCNC(=N)N)C(=O)Nc1ccc2nc(C3=N[C@@H](C(=O)O)CS3)sc2c1]

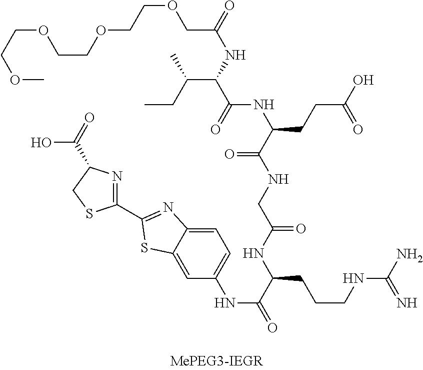 [SMILES: CC[C@H](C)[C@H](NC(=O)COCCOCCOCCOC)C(=O)N[C@@H](CCC(=O)O)C(=O)NCC(=O)N[C@@H](CCCNC(=N)N)C(=O)Nc1ccc2nc(C3=N[C@@H](C(=O)O)CS3)sc2c1]

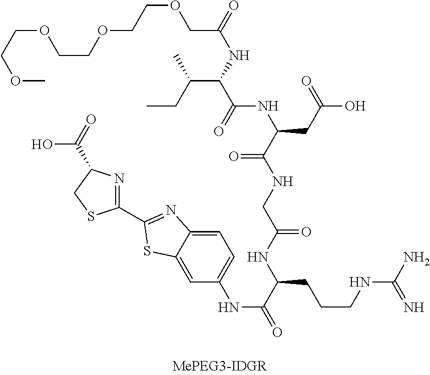 [SMILES: CC[C@H](C)[C@H](NC(=O)COCCOCCOCCOC)C(=O)N[C@@H](CC(=O)O)C(=O)NCC(=O)N[C@@H](CCCNC(=N)N)C(=O)Nc1ccc2nc(C3=N[C@@H](C(=O)O)CS3)sc2c1]

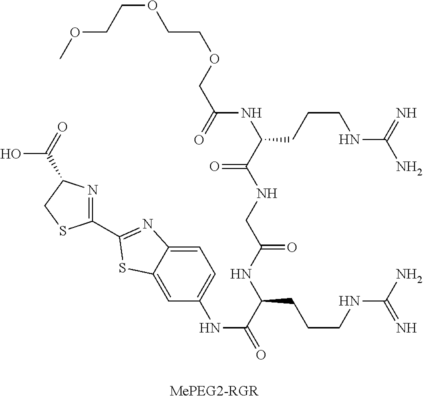 [SMILES: COCCOCCOCC(=O)N[C@H](CCCNC(=N)N)C(=O)NCC(=O)N[C@@H](CCCNC(=N)N)C(=O)Nc1ccc2nc(C3=N[C@@H](C(=O)O)CS3)sc2c1]

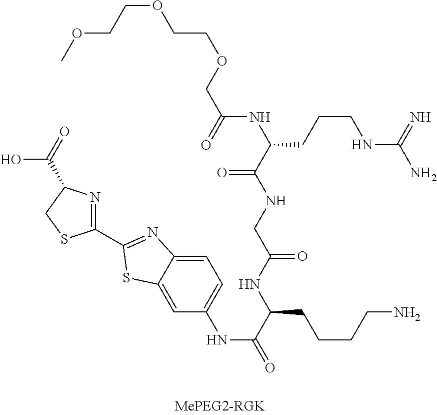 [SMILES: COCCOCCOCC(=O)N[C@H](CCCNC(=N)N)C(=O)NCC(=O)N[C@@H](CCCCN)C(=O)Nc1ccc2nc(C3=N[C@@H](C(=O)O)CS3)sc2c1]

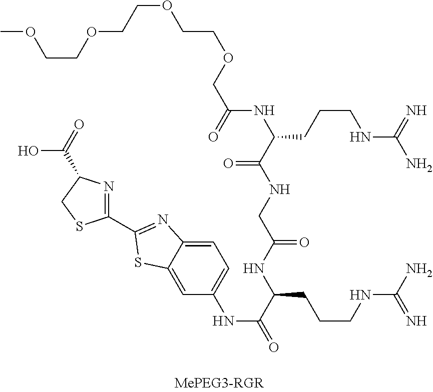 [SMILES: COCCOCCOCCOCC(=O)N[C@H](CCCNC(=N)N)C(=O)NCC(=O)N[C@@H](CCCNC(=N)N)C(=O)Nc1ccc2nc(C3=N[C@@H](C(=O)O)CS3)sc2c1]

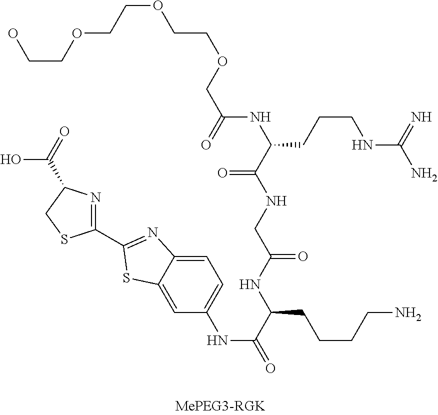 [SMILES: N=C(N)NCCC[C@@H](NC(=O)COCCOCCOCCO)C(=O)NCC(=O)N[C@@H](CCCCN)C(=O)Nc1ccc2nc(C3=N[C@@H](C(=O)O)CS3)sc2c1]

In more preferred embodiments the compound is selected from MePEG2-IEGR, MePEG2-IDGR, MePEG3-IEGR, MePEG3-IDGR, MePEG2-RGR, and MePEG3-RGR. In even more preferred embodiments the compound is selected from MePEG2-IEGR, MePEG2-IDGR, MePEG3-IEGR, and MePEG3-IDGR. In most preferred embodiments is provided the compound according to the invention, wherein it is selected from MePEG2-IEGR and MePEG2-IDGR, preferably MePEG2-IDGR; more preferably a physiologically acceptable salt thereof, such as a TFA salt.

Combination

In another aspect the invention provides a combination comprising a compound according to the invention and at least one further compound selected from the group consisting of luciferase, ATP, an Mg²⁺ source, and a coagulation factor such as factor Xa. Such a combination is referred to hereinafter as a combination according to the invention. Preferably, the combination further comprises luciferase or a coagulation factor such as factor Xa, most preferably it further comprises luciferase. In preferred embodiments, a combination according to the invention comprises each of a compound according to the invention, luciferase, ATP, an Mg²⁺ source, and a coagulation factor such as factor Xa.

Luciferase is a generic term for the class of oxidative enzymes that produce bioluminescence using luciferin as a substrate. Luciferases do not require an external light source, but do require luciferin and O₂, and often also ATP. Mg²⁺ is known to increase luminescent yield of luciferases. Luciferases and their assays are known in the art, and a skilled person can select a suitable luciferase for combination with a luciferin substrate as comprised in a compound according to the invention. The luciferase should be capable of converting the aminoluciferin comprised in the compound according to the invention into its oxidated analogue after the aminoluciferin has been liberated, under emission of a light quant. Suitable luciferases are firefly luciferase (EC 1.13.12.7), Renilla-luciferin 2-monooxygenase (EC 1.13.12.5), and Metridia luciferase (MetLuc). When the compound according to the invention comprises a 2-(6-amino-1,3-benzothiazol-2-yl)-4,5- dihydrothiazole-(4/5)-carboxylic acid moiety, the luciferase is preferably firefly luciferase. The luciferase may be any luciferase known in the art or yet to be discovered or engineered. Many luciferases are known in the art. They can be commercially obtained from manufacturers such as Promega, Sigma, and the like. The luciferase may be a native, a recombinant or a mutant luciferase. Said mutant luciferase may be a modified luciferase comprising one or more amino acid substitutions, amino acid deletions, or amino acid insertions, as long as it retains its luciferase activity, preferably at least 25%, 50%, 75% of the luciferase activity of the native (recombinant) luciferase. It may be derived from any organism, as long as it has luciferase activity. In preferred embodiments, the luciferase is a fast acting luciferase; this is particularly suitable for assays wherein the luciferase should provide quantitative information, because fast acting of luciferase reduces interference that might be caused by luciferase being saturated or outside its linear range.

ATP is required by luciferase and is preferably present in the combination according to the invention in an amount suitable for enabling luciferase activity, or in a stock solution in an amount suitable for preparing dilutions that enable luciferase activity. A suitable ATP stock solution is a 1 mM solution in distilled water, but it can be any stock solution in the range of 200 μM to 10 mM in any physiologically acceptable solvent system.

The combination according to the invention may further comprise magnesium ions as it was found that these magnify the luminescent signal generated by luciferase. However, it is also possible to achieve this effect with other divalent cations such as Mn²⁺ (Rodionova and Petushkov, J. Photochem. Photobiol B., DOI: 10.1016/j.jphotobiol.2005.12.014). An Mg²⁺ source is a source of magnesium ions, which enhances luciferase functioning. Preferred sources of Mg²⁺ are magnesium salts such as magnesium citrate, MgSO₄, MgCO₃, MgO, MgC₁₂, MgF₂, Mgl₂, MgBr₂, and hydrates thereof. Magnesium halides are more preferred, being MgC₁₂, MgF₂, Mgl₂, MgBr₂, and hydrates thereof. A most preferred Mg²⁺ source is MgC₁₂ or a hydrate thereof.

The combination according to the invention can further comprise a coagulation factor. Coagulation factors are sometimes referred to as hemostasis factors and are well-known in the art. Examples of suitable coagulation factors are the group of serine proteases, in particular serine endopeptidases (EC 3.4.21), preferably selected form the group consisting of thrombin, FXa, plasmin, factor Vila, factor IXa, plasma kallikrein, factor XIla, factor Xla, tissue-type plasminogen activator (tPA) (preferably two-chain tPA (tc-tPA)), activated Protein C, and urokinase (uPA) (preferably tc-uPA). Zymogens of serine proteases are also encompassed, such as prothrombin, FX, FVII, FIX, prekallikrein, FXII, FXI, sc-tPA (single-chain tPA), protein C, and sc-uPA. In a highly preferred embodiment, the hemostasis factor is FXa or FX. Preferably, coagulation factors are present in such a combination that FXa can be generated by the factors present, or that FXa can be generated when the combination according to the invention is contacted with a sample comprising a further coagulation factor. In such a case, the sample provides the coagulation factor missing from the cascade to generate FXa, and contact with the sample allows FXa generation. Preferably, all factors present in such a cascade that misses only a single factor are present in excess relative to the expected concentration of the missing factor. This allows the factor from the sample to generate FXa as a function of its concentration, after which FXa can generate a luminescent signal proportional to its concentration, and thus proportional to the concentration of factor in the sample. The section on methods has more details on how such compositions can be constituted.

In preferred embodiments the substances of the combination are comprised in a single composition. Such a composition may comprise further substances such as excipients. Water such as distilled water is a suitable excipient. Other suitable excipients are buffer salts such as Tris (tris(hydroxymethyl)aminomethane).

In other preferred embodiments, the substances of the combination are comprised in distinct compositions. This can be convenient for the provision of kits of parts. In preferred embodiments, the invention provides a kit of parts comprising a compound according to the invention and at least one further compound selected from the group consisting of luciferase, ATP, an Mg²⁺ source, and a coagulation factor such as factor Xa.

In particular embodiments, the invention provides a device for measuring chemiluminescence, the device comprising a compound according to the invention, preferably wherein the device is a point of care device. Such a device is referred to hereinafter as a device according to the invention.

A device according to the invention can for example be a luminometer such as a conventional bench-top luminometer or a luminometer for use in an operation room, or it can be an optical microscope; preferably it is a luminometer. Luminometers and microscopes are known in the art, and a skilled person can select a luminometer or microscope that is suitable for use with the compounds according to the invention. The device according to the invention can also be a disposable or non-disposable cartridge or insert or cuvette or reactor volume comprising a compound according to the invention or a combination according to the invention. Such a device is preferably designed for use with a conventional luminometer or microscope.

In preferred embodiments, the device according to the invention is a point of care device. The small volumes that can be measured using the method according to the invention as described later herein make the method ideally suited to point of care analysis, such as bedside analysis, analysis in the field, or analysis while mobile. A preferred point of care device comprises a mobile luminometer and is suitable for measuring enzyme activity in a sample that is obtained or that has been obtained in the field, or while mobile, or at a bedside.

Preferably a device according to the invention does not comprise a light source for use in analysis. Preferably a device according to the invention has multiple volumes or channels that can be used for concurrent analysis of multiple parameters. Preferably, a device according to the invention is configured to analyse a volume of at most 100, 80, 60, 50, 40, 30, 25, 20, 15, 10, 5, 4, 3, 2, or 1 μL of sample, more preferably of at most 15, 10, 5, 4, 3, 2, or 1 μL, even more preferably of at most 5 μL of sample. Preferably a device according to the invention is configured to present its analysis output in real time, such as via a display screen or via a gauge or level indicator. It is highly preferable when a device according to the invention is configured for simultaneous analysis of at least two different assays, preferably a quantitative assay for enzyme activity such as FVIII activity or FXa activity, and a global assay such as a blood clotting assay.

Method

In a further aspect, the invention provides a method for quantifying a coagulation factor in a sample, the method comprising the steps of:

a) contacting the sample with a composition comprising a compound according to the invention to release aminoluciferin;

b) contacting the aminoluciferin with luciferase; and

c) determining the relative light intensity generated by the luciferase.

Such a method is referred to hereinafter as an assay according to the invention. In this context, quantification of a coagulation factor can be understood as quantification of the activity of a coagulation factor. A skilled person will understand that when activity of a zymogen is determined, the activity of its corresponding enzyme is part of the assay.

The principle of chemiluminescence involving luciferase is well known by the skilled person. It typically uses luciferase, luciferin, ATP, and molecular oxygen for photon production; Mg²⁺ is known to improve luminescence yield of the reaction. Luciferase catalyzes the conjugation of luciferin to ATP, and also the subsequent oxidation of the luciferyl-AMP intermediate. Ultimately, the luciferase provides an environment in which the oxidized luciferin intermediate rearranges to produce oxyluciferin and a single photon with high-quantum efficiency. Light intensity resulting from such luminescence is dependent on the concentrations of the components involved in the chemical or enzymatic conversion of the liberated luminescent molecule. By using an excess of such components, the luminescent signal of the method of the invention becomes dependent only on the generation of free luminescent molecules by cleavage of the substrate by the hemostasis factor of interest, e.g., FXa or another factor as later described herein. Under such circumstances, the light intensity thus is proportional to the generation of said hemostasis factor, e.g., FXa generation. Thus the coagulation factor of interest can be quantified through design of the assay according to the invention, as its concentration is proportional to the light output of the assay.

The inventors found that by using compounds according to the invention, generation of hemostasis factor, e. g. FXa generation be it direct or indirect, or FXa concentration as such, can be measured continuously, semi-continuously, or in a direct way without requiring calculation of the first derivative as is required for chromogenic or fluorogenic method for measuring generation of blood clotting factors. Typically, upon cleavage of a substrate of the invention by a hemostasis factor of interest, a ‘luminescent molecule’ is liberated, being the aminoluciferin, which is prone to a subsequent chemical or enzymatic conversion that produces a detectable light signal (or “light quant” or “photon” or “light unit”). Since the light quant is produced in an irreversible step, there is no accumulation of output signal; the signal is a direct measure of FXa activity (and thus possibly of the activity of a factor involved in FXa generation). It is a significant advantage of the present method, as compared to existing methods employing fluorescent or chromogenic substrates, that a signal can be detected real-time that is directly proportional to the amount of the hemostasis factor present at any given time point; there is no need to calculate the first derivative of an accumulating optical signal. In the present method there is no interference with further production of light signals. In addition, no external light source and optical filters are required for measuring the signal. Thus, the method of the present invention is more convenient than the prior art methods.

A sample can be a sample from a subject, preferably it is a sample that has been previously obtained from a subject. A subject can be a human. A subject can be non-human. A sample is preferably a fluid. A preferred sample is blood or derived from blood. Suitable samples are whole blood and plasma such as platelet poor plasma or platelet rich plasma. A most preferred sample is platelet poor plasma, such as platelet poor plasma that has been previously obtained from a subject.

Coagulation factors are well known in the art. In the context of the assay according to the invention, preferred coagulation factors are factor IX (FIX), factor IXa (FIXa), factor VIII (FVIII), factor ViIIa (FVIIIa), factor VII (FVII), factor VIIa (FVIIa), factor XI (FXI), factor Xla (FXIa), Factor XII (FXII), factor XIIa (FXIIa), factor X (FX), and factor Xa (FXa). A notation such as FX(a) references both or either of FX and FXa.

A physiological role of coagulation factors is to ultimately enable thrombin generation. Thrombin is the most important constituent of the coagulation cascade in terms of its feedback activation roles. In humans parts of the process are generally as follows: FVIIa circulates in a higher amount than any other activated coagulation factor. Following damage to the blood vessel, FVII(a) can come into contact with tissue factor (TF), ultimately forming an activated complex (TF-FVIIa). TF-FVlla activates FIX to form FIXa, and activates FX to form FXa. The activation of FX (to form FXa) by TF-FVIIa is almost immediately inhibited by the TF (tissue factor) pathway inhibitor (TFPI) in the TF-TFPI-FXa complex. FXa and its co-factor FVa form the prothrombinase complex, which activates prothrombin to thrombin. Thrombin then activates other components of the coagulation cascade, including FV and FVIII (which forms a complex with FIX), and activates and releases FVIII from being bound to vWF, forming FVIIIa. FVIIIa is the co-factor of FIXa, and together they form the “tenase” complex, which activates FX, continuing the cycle.

In preferred embodiments is provided the assay according to the invention, wherein the coagulation factor is selected from factor IX, factor IXa, factor VIII, factor Villa, factor VII, factor Vila, factor XI, factor Xla, Factor XII, factor Xlla, factor X, and factor Xa. The coagulation factor should be FXa or should contribute to formation of FXa.

For example, when the coagulation factor to be assayed is FXa, then the coagulation factor is FXa. When the coagulation factor to be assayed is not FXa, then the coagulation factor should contribute to formation of FXa. For this, FX should be present.

In certain combinations the generation of FXa is proportional to the concentration of several coagulation factors like FVIII, FIX and FX in the tenase complex, or tissue factor or FVlla in the TF-FVlla complex. When all factors but a missing one are present in excess, then the activity of the missing factor can be correlated to eventual FXa activity as determined via luminescence output.

FX can be converted into FXa by FVlla or by FVlla in the presence of TF, so when FVlla is to be assayed, the coagulation factor should be FX; this is because the FVlla in the sample can then convert an excess of FX into FXa.

FX can also be converted into FXa by FIXa and FVIIIa, in a so-called tenase complex. A tenase complex consists of FIXa, FVIIIa, FX, anionic phospholipids, and calcium. Accordingly, when FIXa is to be assayed, the coagulation factor should be FX or FVIIIa, and preferably both FX and FVIIIa should be present, more preferably along with anionic phospholipids and calcium. Accordingly, when FVIIIa is to be assayed, the coagulation factor should be FX or FIXa, and preferably both FX and FIXa should be present, more preferably along with anionic phospholipids and calcium. In turn, FIX can be converted into FIXa by FXla, so when FIX is to be assayed, FXIa is preferably present in addition to the conditions described for FIXa. A skilled person is aware of the various interactions in coagulation pathways and will be capable of selecting suitable coagulation factors to be present in an assay according to the invention, depending on which coagulation factor is to be assayed. In general, components of the coagulation pathway are to be present in excess, with the component to be assayed omitted. The coagulation factor to be assayed is then supplied via the sample, and because any of its substrates, cofactors, or other interactors are present in excess, the amount of coagulation factor to be assayed will directly correlate to the FXa activity that is generated and that is ultimately detected via the compounds according to the invention.

A skilled person will understand that detection of a zymogen will generally involve detection of the corresponding enzyme, and that a detection of the zymogen thus amounts to detection of both the zymogen and the corresponding active enzyme. For example, detection of FVII results in simultaneous detection of FVII and FVlla.

Global assay

In one class of preferred embodiments, all components of the coagulation pathway are present. Such an assay is referred to herein as a global assay, and it is preferably preformed using the coagulation factors provided by the sample itself. Accordingly, in a global assay, preferably the components of the coagulation pathway are present in ratios that resemble the ratios found in physiological systems. Preferred samples for a global assay are whole blood and plasma such as platelet rich plasma, or platelet poor plasma, more preferably plasma, most preferably platelet poor plasma. A global hemostasis assay is preferably initiated by addition of active coagulation factors such as FXlla, FXla, or FIXa, of tissue factor (TF) and/or of calcium to plasma, more preferably of TF and/or of calcium, most preferably of both TF and calcium; this leads to FXa generation followed by thrombin generation and subsequent clot formation. Following initiation, thrombin is required for propagation and termination of the cascade. Measuring the FXa concentration in such a global design gives information about the sample's capacity of the coagulation cascade.

Step a)—Release of Aminoluciferin

In step a) the sample is contacted with a composition comprising a compound according to the invention to release aminoluciferin. FXa is able to recognize the peptide comprised in the compound according to the invention, and cleaves it from the luminescent moiety to release aminoluciferin. Accordingly, when FXa is the coagulation factor to be assayed, no other coagulation factors need to be present during step a). In highly preferred embodiments, calcium and phospholipids are present during step a). The calcium and phospholipids should be suitable for formation of a tenase complex. These phospholipids are preferably anionic phospholipids.

Conditions for coagulation factor activity are known in the art, and it is under these circumstances that the contacting is preferably done. An example is the use of a physiologically acceptable buffer, for example Tris buffer optionally comprising 1% serum albumin such as BSA. An example of a suitable Tris buffer is Tris buffered saline (TBS; 50 mM Tris—CI, 150 mM NaCI; pH 7.4). The concentration of the substrate according to the invention is preferably at least 1, 2, 3, 4, 5, 6, 7, 8, 9, 10, 15, 20, 25, 30, 35, 40, 45, or 50 μM or more, more preferably at least 20 or 30 μM such as at least 30 μM. The concentration of the substrate according to the invention is preferably at most 5000, 4000, 3000, 2000, 1750, 1500, 1250, 1000, 900, 800, 700, 600, 500, 400, 300, 200, 100, 90, 80, 70, 60 μM or lower, more preferably at most 2500 or 200 μM or 1750 μM or 1500 μM or 1250 μM or 1000 μM or 900 μM or 800 μM or 700 μM or 600 μM or 500 μM or lower, even more preferably at most 2000 or 750 μM or lower, such as at most 750 μM.

This step is to correlate the presence of coagulation factor to be assayed to a luminescent output, and accordingly a substrate for luciferase is released in this step. In this context, release is to be interpreted as a hydrolysis of for example the aminoluciferin moiety as shown in general formula II-4 wherein the carboxylic acid is S. Depending on the substrate, other luminescent moieties can also be released. The release is to make the chemiluminescent substrate available for subsequent conversion in step b), as the chemiluminescent substrate is not available for further conversion when it is comprised in a compound according to the invention. In preferred embodiments is provided the assay according to the invention, wherein the coagulation factor to be assayed is selected from factor IX, factor IXa, factor VIII, factor Villa, factor VII, factor Vila, factor XI, factor Xla, Factor XII, factor Xlla, factor X, factor Xa, prekallikrein, and kallikrein, preferably selected from factor IX, factor IXa, factor VIII, factor Villa, factor X, and factor Xa,

optionally wherein during step a) the composition comprises at least one further coagulation factor selected from factor IX, factor IXa, factor VIII, factor Villa, factor VII, factor VIIa, factor XI, factor Xla, factor XII, factor Xlla, factor X, prekallikrein, and kallikrein, preferably selected from factor IX, factor IXa, factor VIII, factor Villa, and factor X. The coagulation factor should be FXa or should contribute to formation of FXa as described above. The further coagulation factor should contribute to the generation of FXa.

FX can be converted into FXa by FIXa and FVIIIa, in a so-called tenase complex. A tenase complex consists of FIXa, FVIIIa, FX, anionic phospholipids, and calcium. Accordingly, when FIXa is to be assayed, the coagulation factor can be either one of FX or FVIIIa, and the further coagulation factor is then preferably either FX or FVIIIa, depending on which factor is not the coagulation factor already selected. Accordingly, when FVIIIa is to be assayed, the coagulation factor should be either one of FX or FIXa, and the further coagulation factor should be preferably either FX or FIXa, depending on which factor is not the coagulation factor already selected. A skilled person is aware of the various interactions in coagulation pathways and will be capable of selecting suitable coagulation factors and further coagulation factors to be present in an assay according to the invention, depending on which coagulation factor is to be assayed.

Step b)—Oxidation of Aminoluciferin by Luciferase

In step b) the aminoluciferin is contacted with luciferase. The purpose of this contacting is to generate a light quant from the chemiluminescent molecule that was released in step a) such as from the released aminoluciferin. Methods for converting a chemiluminescent substrate to produce a light quant are established in the art, and the luciferase, preferably firefly luciferase, can become more functional when further substances are also present during this contacting. Accordingly, in preferred embodiments, step b) further comprises contacting the released chemiluminescent molecule with ATP. In preferred embodiments, step b) further comprises contacting the released chemiluminescent molecule with Mg²⁺. In highly preferred embodiments, step b) further comprises contacting the released chemiluminescent molecule with ATP and Mg²⁺. When ATP is also present during step b), it can be present at a final concentration of about 50-1000 μM, preferably at a final concentration of about 250-500 μM, more preferably of about 300-400 μM such as about 333 μM. When Mg2+ is present during step b), it can be present at a final concentration of about 1-30 μM, it is preferably present at a final concentration of about 4-12 mM, more preferably of about 6-10 mM, such as about 8.3 mM. Luciferase is preferably present at about 0.05-50 mg/mL, more preferably at about 0.1-10 mg/mL, even more preferably at about 0.5 to 5 mg/mL such as at about 0.9 mg/mL.

Step a) and step b) can be performed simultaneously and/or in the same reaction volume. In preferred embodiments, step a) and step b) are performed in the same reaction volume, which preferably simultaneously comprises all reagents required for both steps a) and b). This allows a released chemiluminescent molecule to be converted by luciferase without delay.

Step c)—Determining the Relative Light Intensity

In step c) the luminescent signal is determined. This can be done in any way that is known in the art, for example using a luminometer. The determined light intensity is used as a basis for quantifying the coagulation factor to be assayed. This is because, as described earlier herein and as exemplified in examples 5 and 6, the concentration of the coagulation factor correlates to the relative light intensity, preferably expressed as relative light units (RLU). In some embodiments, the relative light intensity is compared to a reference value or to a calibration curve. Such a calibration curve has preferably been prepared using known amounts of the coagulation factor to be assayed, for example as demonstrated in the examples. A reference value can be a set value such as a predetermined value, or it can be the assay result from a control sample. In this context a control sample is preferably a sample that is known to meet certain specifications, or a sample (previously)

obtained from a healthy subject, or it is normal pooled plasma. The relative light intensity directly represents the actual luciferase activity, which in turn directly represents the actual amount of luminescent substrate being released, which in turn directly represents FXa activity. As described above, FXa activity can in turn directly represent the activity of other coagulation factors. The relative light intensity thus provides direct information about the amount of coagulation factor to be assayed, owing to the fixed ka of the enzymes and substrates involved. This is in contrast to accumulating assays such as fluorogenic assays or chromogenic assays. For the latter two assays the slope must be calculated to determine conversion rate. In luminescent assays the flat output (for example in RLU) directly indicates the conversion rate. In preferred embodiments, step c) does not comprise determining a derivative of any signal determined in step c). In more preferred embodiments, step c) does not comprise determining the first derivative of the relative light intensity generated by the luciferase. In this context, light intensity generated by the luciferase relates to the relative light intensity resulting from the luminescent molecule such as aminoluciferin being released from the compound according to the invention. Luminescence can be measured at wavelengths according to methods known in the art. Examples of suitable wavelengths are wavelengths between 360-630 nm.

The relative light intensity as determined at a point in time thus directly provides relevant information about coagulation factor activity. Still, determination of relative light intensity during a set duration of time, or until a certain condition is met, can provide relevant information about the dynamics of an assay. This is particularly useful for a global assay as described above, wherein the output will generally be a bell-shaped curve. Accordingly, in preferred embodiments, step c) comprises determining the relative light intensity generated by the luciferase over a period of time.

This period of time is preferably at most 150, 120, 90, 80, 70 , 60, 55, 50, 45, 40, 35, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 minute or shorter, more preferably at most 35, 30, 15, or 10 minutes or shorter. A period of time is preferably at least 1, 2, 3, 4, 5, 6, 7, 8, 9, 10, 15, 20, 25, 30, 35, 40, 45, 50, 55, or 60 seconds, more preferably at least 10, 20, or 30 seconds such as at least 30 seconds.

Method for quantifying an anticoagulant in a sample

The assay according to the invention can be modified to allow quantification of anticoagulants. Accordingly, the invention also provides a method for quantifying an anticoagulant in a sample, the method comprising the steps of:

a) contacting the sample with a composition comprising factor Xa and a compound according to the invention to release aminoluciferin;

b) contacting the aminoluciferin with luciferase; and

c) determining the relative light intensity generated by the luciferase. This method is a method that quantifies inhibition of FXa activity, and it is referred to hereinafter as an inhibition assay according to the invention. A difference with the assay according to the invention as described above is that in this method FXa is also provided in step a). A result of this is that a known amount of luminescent signal will be generated when no inhibitor of FXa is present, as a result of FXa activity releasing aminoluciferin from the compounds according to the invention.

Therefore any reduction in luminescent output can be ascribed to FXa inhibition, such as when an anticoagulant is present. Preferred anticoagulants to be assayed are direct acting anticoagulants (DOACs), heparin, and heparinoids. Preferred DOACs are against FXa, such as rivaroxaban (CAS 366789-02-8), apixaban (CAS 503612-47-3), and edoxaban (CAS 480449-70-5).

As discussed, FXa is already provided in step a). This provision of FXa can be direct provision of FXa, or it can be provision of a composition that can generate FXa. Such compositions are known in the art. Preferably, when stap a) comprises provision of a composition able to generate FXa, the composition generates a fixed or known amount of FXa.

Steps b) and c) do not materially differ and accordingly have been described above. For step c) as used in this method for quantification of anticoagulants comparison to a reference value can be preferred, particularly when the reference value is a calibration curve wherein FXa inhibition by known amounts of the anticoagulant to be assayed have been used. This is exemplified in Example 4 and in FIG. 4.

In preferred embodiments, the invention provides an assay according to the invention or an inhibition assay according to the invention, wherein step c) comprises determining the relative light intensity generated by the luciferase over a period of time, and/or wherein the assay or inhibition assay does not comprise determining the first derivative of the relative light intensity generated by the luciferase.

Altogether, the present invention provides an improved, sensitive method for monitoring generation of hemostasis factors in a test sample. The method of the present invention allows for the design of an optical point-of-care device for measuring the generation of one or more hemostasis factors.

General Definitions

In preferred embodiments, compounds and compositions according to the invention are for use in methods according to the invention, or are for use according to the invention. Each embodiment as identified herein may be combined together unless otherwise indicated.

When a structural formula or chemical name is understood by the skilled person to have chiral centers, yet no chirality is indicated, for each chiral center individual reference is made to all three of either the racemic mixture (having any enantiomeric excess), the pure R enantiomer, and the pure S enantiomer.

Compounds and compounds for use provided in this invention can be optionally substituted. Suitable optional substitutions are replacement of —by a halogen. Preferred halogens are F, CI, Br, and I. Further suitable optional substitutions are substitution of one or more —by —NH2, —OH, =0, alkyl, alkoxy, haloalkyl, haloalkoxy, alkene, haloalkene, alkyne, haloalkyn, and cycloalkyl. Alkyl groups have the general formula CₙH₂ₙ₊₁ and may alternately be linear or branched. Unsubstituted alkyl groups may also contain a cyclic moiety, and thus have the concomitant general formula CₙH_{2n-1.} Optionally, the alkyl groups are substituted by one or more substituents further specified in this document. Examples of alkyl groups include methyl, ethyl, propyl, 2-propyl, t-butyl, 1-hexyl, 1-dodecyl, etc. Throughout this application, the valency of atoms should always be fulfilled, and H can be added or removed as required. Most preferably, optional substitutions are substitution of one, two, or three —H by F, CI, CH₃, CH₂CH₃, OCH₃, or OCH₂CH₃.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such a charge more often than that it does not bear or carry such a charge. As such, an atom that is indicated in this disclosure to be charged could be non-charged under specific conditions, and a neutral moiety could be charged under specific conditions, as is understood by a person skilled in the art.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

In this document and in its claims, the verb “to comprise” and its conjugations is used in its non-limiting sense to mean that items following the word are included, but items not specifically mentioned are not excluded. “Hemostasis” and “Haemostasis” can be used interchangeably herein. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 1% of the value. In addition, the verb “to consist” may be replaced by “to consist essentially of” meaning that a composition of the invention may comprise additional component(s) than the ones specifically identified, said additional component(s) not altering the unique characteristics of the invention.

All patent and literature references cited in the present specification are hereby incorporated by reference in their entirety.

In the context of this invention, a cell or a sample can be a cell or a sample from a sample obtained from a subject. Such an obtained sample can be a sample that has been previously obtained from a subject. Such a sample can be obtained from a human subject. Such a sample can be obtained from a non-human subject.

The following are sequences referred to in this invention:

SEQ ID NO: 1
IEGR
SEQ ID NO: 2
IDGR
SEQ ID NO: 3
IEGK
SEQ ID NO: 4
IDGK

### SHORT DESCRIPTION OF DRAWINGS

FIG. 1-13 Synthesis of compounds according to the invention. A) synthesis towards a shared Gly-Arg/Gly-Orn core; B) conversion of the shared core into a precursor having peptide core Ile-Glu-Gly-Arg; C) conversion of the Ile-Glu-Gly-Arg intermediate into MePEG2-IEGR.

FIG. 2—Michaelis-Menten kinetics for the enzyme FXa and its substrate MePEG2-IEGR at an FXa concentration of 1.6 nM. A Vₘₐₓ=468000 RLU and a K_{M}=619 μM were determined, leading to a K_{cat}=Vₘₐₓ/[FXa]=1.05×10¹⁴ s⁻¹.

FIG. 3—Substrate MePEG2-IEGR has minimal cross-reactivity with serine proteases other than FXa. A) 80 nm FXa yielded a signal of 1.000.000 RLU; 52 nM thrombin yielded ˜30.000 RLU; 8 nM plasmin yielded ˜8.000 RLU; 193 IU/mL tPA (the conventional concentration to facilitate fibrinolysis) yielded ˜6.000 RLU; 145 nM FXlla yielded ˜500 RLU; concentrations other than tPA are representative for concentrations in human plasma after activation. B) closer view of a section of panel A wherein the y-axis has been transformed to a logarithmic scale.

FIG. 4—Apixaban inhibits the conversion of substrate MePEG2-IEGR. FXa (4 nM) in the presence of 100 pg/mL to 100 mg/mL Apixaban reveals inhibition in the 10-1000 ng/ml range.

FIG. 5—FVIII activity in a sample was determined based on FXa activity by providing a reaction mixture comprising excess components of cascade participants but lacking FVIII. Luminescence as caused by FXa activity thus becomes a measure of FVIII activity; A) RLU measured at different FVIII concentrations expressed as percentage of normal pooled plasma; B) calibration line for the results in panel A. The calibration has a linear regression of y=30331+4376x and r2=0.9935.

FIG. 6—FIX activity in a sample was determined based on FXa activity by providing a reaction mixture comprising excess components of cascade participants but lacking FIX. Luminescence as caused by FXa activity thus becomes a measure of FIX activity; A) RLU measured at different FIX concentrations expressed as percentage of normal pooled plasma; B) calibration line for the results in panel A. The calibration has a linear regression of y=97471+8547x and r²=0.9872.

FIG. 7—FXa activation in a global hemostasis assay using high and low tissue factor concentration (7 μM or 0.28 μM).

FIG. 8—A) Raw data measured kinetically with a chemiluminescent reader. B) This example uses a fixed time point, in this case 3 minutes, to construct a calibration curve. C) This example calculates the slope between 0.5-3 minutes of each calibrator to construct the calibration curve.

FIG. 9-13 A) An overlay of 4 calibration curves utilized to measure 31 FVIII clinical samples. The average R² value of these four calibration curves combined was 0.9810. B) Recovery of the clinical samples in both the chemiluminescent-based assay as well as the chromogenic FVIII assay using samples containing a recombinant PEGylated FVIII product. C) As for B), but using samples containing a recombinant full-length FVIII product.

FIG. 10-13 A) Raw data measured kinetically with a chemiluminescent reader with the calibrators containing various FIX levels. B) Constructed calibration curve.

### EXAMPLES

### Example 1

### Provision of Substrates According to the Invention

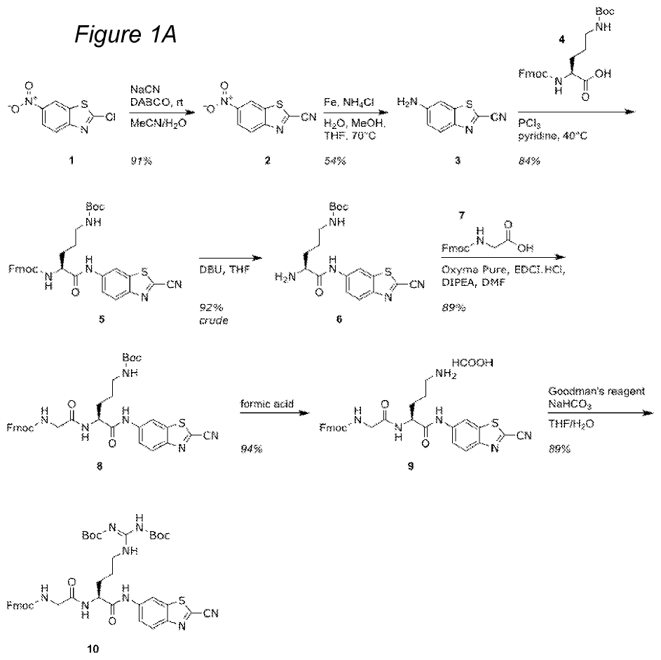

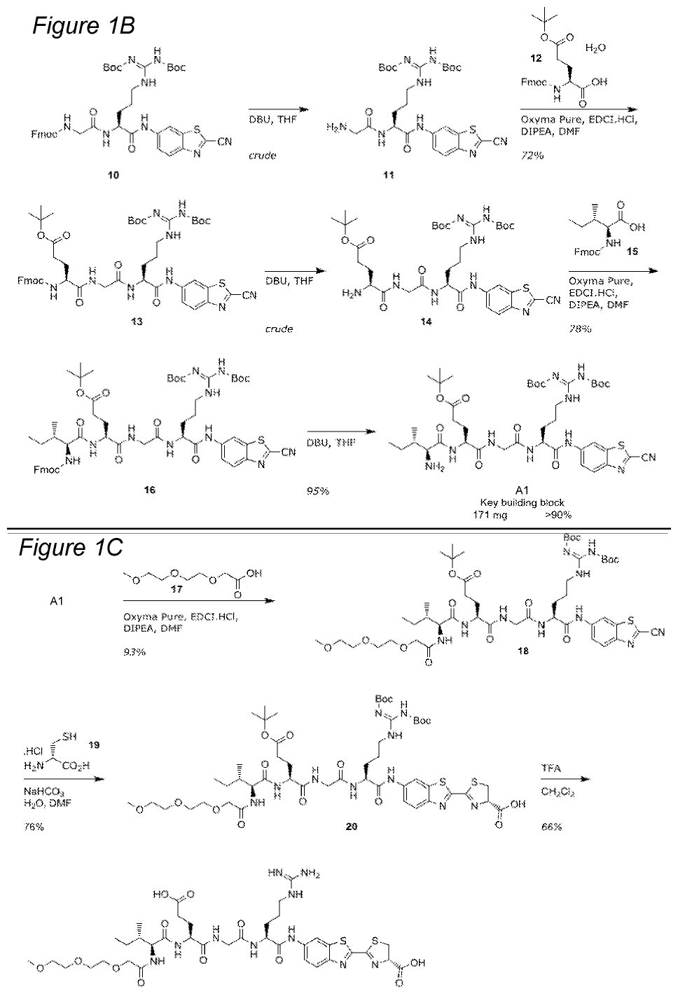

As shown in FIG. 1A, substrates were synthesized via key intermediate A1. The synthesis of this intermediate started with the conversion of benzothiazole 1 towards nitrile 2 in 91% yield. Reduction afforded pure aniline 3 in 54% yield, after straight phase and reversed phase flash column chromatography. The coupling with Fmoc-Orn(Boc)-OH (4) was performed with PCI₃ in pyridine. After Fmoc-deprotection compound 6 was obtained successfully. Peptide coupling of 6 with Fmoc-Gly-OH 7 towards 8 and Boc-deprotection gave 9 in 84% yield over these two steps. The reaction with 1,3-bis(tert-butoxycarbonyI)-2-(trifluoromethanesulfonyl)guanidine gave guanidine 10 in good yield and purity after purification by flash column chromatography. For provision of compounds of general formula I-3 or I-4 wherein other amino acids are present, the above protocol is repeated with said other amino acids, or compound 8 is not converted into compound 10.

As shown in FIG. 1B, Fmoc-deprotection and immediate further coupling with Fmoc-Glu(OtBu)-OH 12 gave 13 in 72% yield over 2 steps. Repeating this sequence, but this time using Fmoc-Ile-OH 15, gave tetrapeptide 16 in 78% yield over 2 steps. Fmoc-deprotection to key building block Al went in 95% yield. For provision of compounds of general formula I-3 or I-4 wherein other amino acids are present, the above protocol is repeated with said other amino acids.

As shown in FIG. 1C, the synthesis was continued with the peptide coupling of A1 with [2-(2-methoxyethoxy)ethoxy]acetic acid 17 towards 18 in 93% yield. For other moieties X similar couplings can be performed using the appropriate reagents. The coupling with D-cysteine 19 gave aminoluciferin 20 in 76% yield after purification by reversed phase preparative chromatography. The removal of the protecting groups was performed by using trifluoroacetic acid, after which the product, designated MePEG2-IEGR, was isolated by trituration in diethyl ether.

### Example 2

### Kinetic Behaviour of Substrates According to the Invention

Michaelis-Menten kinetics were determined for the enzyme FXa and its substrate MePEG2-IEGR (the TFA salt was used in these examples). The following composition was prepared in a microvial on ice:

### Composition 1:

160 μL Tris buffered saline (TBS) 50 mM Tris-HCI, 150 mM NaCI (pH 7.4)

4 μL ATP (final concentration 333 μM)

4 μL MgC₂ (final concentration 8.3 mM)

12 μL Luciferase (final concentration 0.9 mg/ml)

Substrate compositions 2a-g:

Microvials were prepared with 30 μL substrate concentrations leading to final concentrations of 2000, 1333, 1000, 666, 333, 167 and 66 μM.

Enzyme composition 3:

FXa (Coachrom, 16 nM) composition was prepared in a microvial on ice

Subsequently, the following compositions were added together in a white 384-well plate:

24 μL Composition 1

3 μL Composition 2a-g

3 μL Composition 3

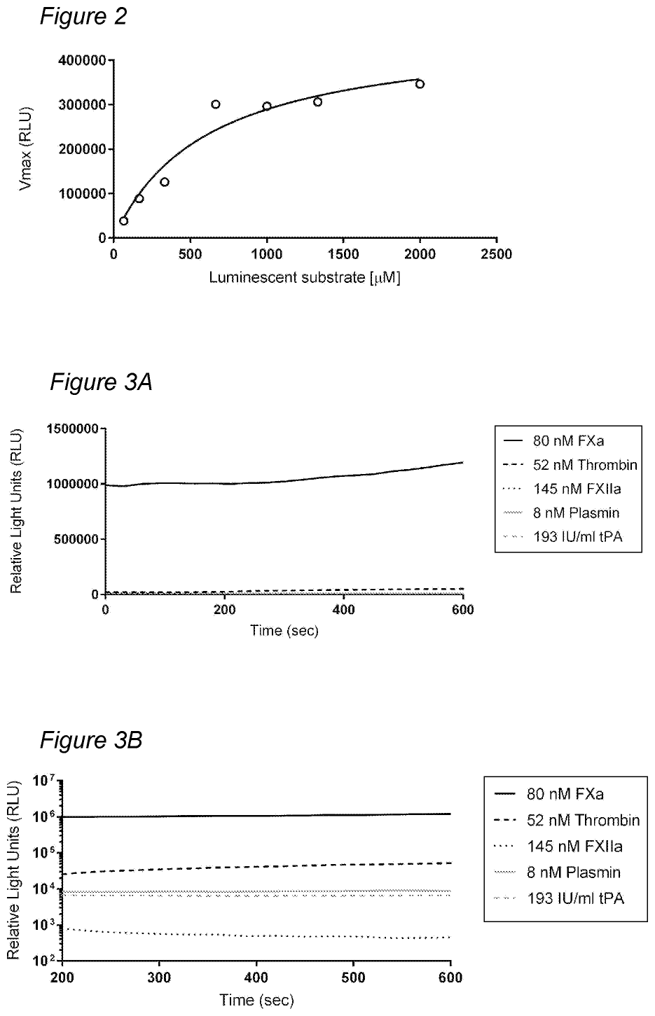

Subsequently the well plate was placed in a 37 ° C. thermostated Flexstation 3 (Molecular Devices). Luminescence emission wavelength was with an integration time of 1500 ms, measured for 1s every 30 s during 30 min.
Results are shown in FIG. 2. At an FXa concentration of 1.6 nM a Vₘₐₓ=468000 RLU and a K_{M}=619 μM were determined, leading to a K_{cat}=Vₘₐₓ/[FXa]=1.05×10¹⁴ s⁻¹.

### Example 3

### Cross Reactivity

To determine cross-reactivity an assay was performed using a range of coagulation enzymes at representative concentrations in human plasma. The chosen concentrations of the enzymes are comparable to the expected concentrations in vivo after activation. For tissue-type plasminogen activator (tPA) this is the conventional concentration for facilitation of fibrinolysis.
A concentration of 80 nm FXa yielded a signal of 1.000.000 RLU. Other coagulation enzymes resulted in the following signals: 52 nM thrombin yielded ˜30.000 RLU, 8 nM plasmin yielded ˜8.000 RLU, 193 IU/mL tPA yielded ˜6.000 RLU and 145 nM FXlla yielded ˜500 RLU (FIG. 3). These data indicate that the substrate according to the invention is preferentially cleaved by FXa.

### Example 4

### Quantification of Anticoagulant Activity

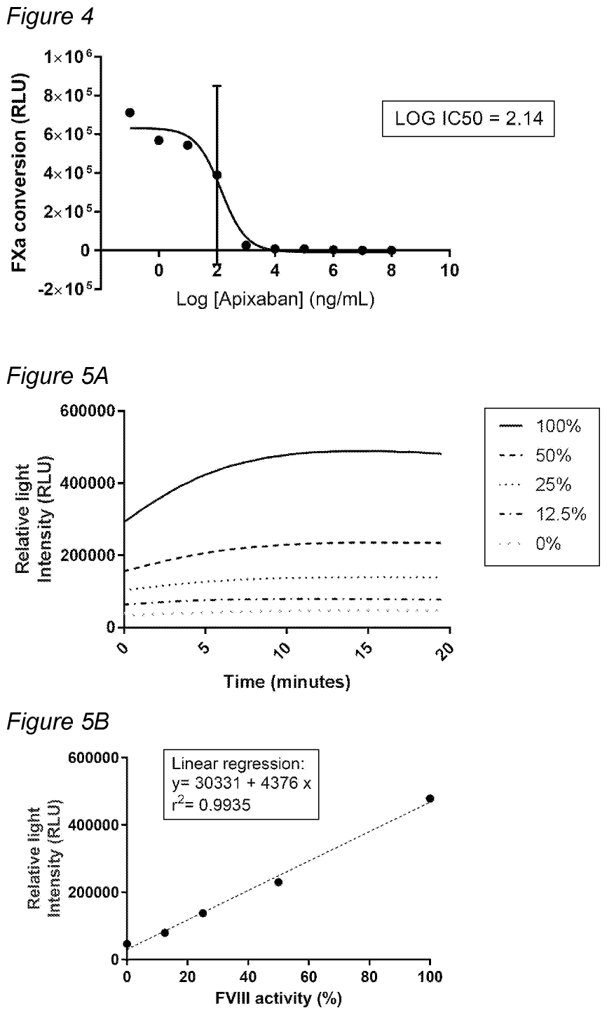

As an example of an anticoagulant, apixaban (CAS Number 503612-47-3) was added at different concentrations into a mixture of FXa and MePEG2-IEGR. Apixaban is an anticoagulant for the treatment of venous thromboembolic events, to be taken orally. It is a direct FXa inhibitor. FIG. 4 shows the inhibition of apixaban on the conversion of the substrate by FXa (4 nM) in the presence of 100 pg/mL up to 100 mg/mL apixaban. Inhibiting effect is shown on FXa activity in the 10-1000 ng/ml range.

### Example 5

### Quantitative FVIII Assay

This luminescent FVIII assay is a two-step activity assay that leads to activation of a fixed amount of FVIII from a sample, which subsequently leads to FX activation and thus to stable conversion of the substrate according to the invention. Luminescent factor assays lead to stable substrate conversion due to the fast decay time of the photons, whereas chromogenic or fluorescent substrates accumulate signal (at e.g. 405 nm), leading to increased signal over time (OD/min). For the latter assays the slope must be calculated to determine conversion rate. In luminescent assays the flat output (in RLU) directly indicates the conversion rate, which makes the methods of the present invention very convenient.
In the assay thrombin is added to activate FVIII. Activated Factor VIII forms an enzymatic complex with Factor IXa, phospholipids (PLPs), and calcium. This complex activates Factor X to FXa; FX is supplied in the assay at a constant concentration and in excess. FXa acts on the substrates according to the invention to liberate a substrate for luciferase, leading to luminescent activity. The luminescent activity is thus directly related to the amount of Factor VIII activity, which is the limiting factor in the presence of a constant amount of Factor IXa. Generated Factor Xa is then exactly measured by its activity on the specific Factor Xa luminescent substrate of the invention. Factor Xa cleaves the substrate and leads to release of a photon. The amount of photons generated (expressed as Relative Light Units, RLU) are directly proportional to the Factor Xa activity and consequently the amount of Factor VIII. Chromogenic analogues of such assays are commercially available (for example BIOPHEN Factor VIII assay from Hyphen Biomed, Catalogue Ref. 221402). An important difference with this example is that the substrate according to the invention is used with ATP, Mg²⁺, and luciferase, instead of a chromogenic substrate.
Different amounts of FVIII (using normal pooled plasma (NPP) or dilutions thereof) were used in the assay. The samples were mixed with a solution of FX. After about 2 minutes of incubation at 37° C. a solution of FIXa, thrombin, calcium, and phospholipids in distilled water was added, after which the reaction was incubated for about 3 minutes at 37° C. Then MePEG2-IEGR (in this case the TFA salt) was added as substrate for FXa, after which luminescence was determined using a conventional mixture of luciferase, ATP, and Mg²+. FIG. 5A shows the RLU of FXa activity dependent on the FVIII activity present in the sample, with added FVIII expressed as NPP percentage. FIG. 5B shows the concomitant calibration line. The calibration has a linear regression of y=30331+4376 x and r²=0.9935.

### Example 5.1

### Detailed FVIII Assay

### Reagents

### Reagent 1 (R1):

Diluted plasma samples used for testing or for the calibration curve. Samples are diluted with buffer containing 50 mM imidazole and 100 mM NaCI (pH 7.4).

### Reagent 2 (R2):

Human Factor IXa, human thrombin, calcium, Gly-Pro-Arg-Pro as fibrin polymerization inhibitor and synthetic phospholipids (28% phosphatidylserine). The phospholipids and calcium are stored at 4-8° C. Other components are stored at −80° C. The reagent mixture is prepared in buffer containing 50 mM imidazole and 100 mM NaCI (pH 7.4).

### Reagent 3 (R3):

Human Factor X, ATP, magnesium, luminescent substrate specific for Factor Xa (in this example, MePEG2-IEGR was used) and the recombinant Luciferase Ultra-Glo from Promega. The luciferase can be exchanged for the QuantiLum luciferase, both types can be applied. Magnesium is stored at 4-8° C. The individual components are stored at −80° C. in aqueous solution. The reagent mixture is prepared in buffer containing 50 mM imidazole and 100 mM NaCI (pH 7.4).

### Preparation Calibration Curve Samples

Table 5 shows the preparation of calibration samples for the FVIII assay. For the preparation of the calibration curve, the following diluted plasma samples are mixed as is shown in table 5. In the 384-well assay plate, 3 μL of the diluted sample or calibrator is used for the 10 μL test volume. Unknown specimens are prepared the same as the 100% samples.

TABLE 5

| | Stock solution | | | | | Working solution | | | |
|---|---|---|---|---|---|---|---|---|---|
| | Start | | Volume | | | | | | Final |
| | conc. | Volume | FVIIIdef | Total | | Volume | Volume | Total | conc. |
| Cali- | (FVIII | start | plasma | volume | | stock | buffer | volume | (FVIII |
| brator | %) | (μL) | (μL) | (μL) | Dilution | (μL) | (μL)^{*} | (μL) | %) |
| C1 | 100 | 20 | 0 | 20 | 7/30 | 3.5 | 11.5 | 15 | 100 |
| C2 | 100 | 10 | 10 | 20 | 7/30 | 3.5 | 11.5 | 15 | 50 |
| C3 | 50 | 10 | 10 | 20 | 7/30 | 3.5 | 11.5 | 15 | 25 |
| C4 | 25 | 10 | 10 | 20 | 7/30 | 3.5 | 11.5 | 15 | 12.5 |
| C5 | 12.5 | 10 | 10 | 20 | 7/30 | 3.5 | 11.5 | 15 | 6.25 |
| C6 | 6.25 | 10 | 10 | 20 | 7/30 | 3.5 | 11.5 | 15 | 3.125 |
| C7 | 3.125 | 10 | 90 | 100 | 7/30 | 3.5 | 11.5 | 15 | 0.3 |

^{*}buffer is 50 mM imidazole and 100 mM NaCl.

### Preparation Protein Reagents

Tables 6 and 7 describe the preparation of reagent 2 or 3, respectively. These volumes represent one reaction or one sample in a 384 wells plate with a final test volume of 10 μL or 30 μL. The final concentration in each well is described in table 8.

TABLE 6

| | | Volume in | Volume in |
|---|---|---|---|
| Component | Stock conc. | 10 μL (μL) | 30 μL (μL) |
| FIXa (human) | 274 nM | 0.25 | 0.75 |
| Phospholipids | 0.5 mM | 0.2 | 0.6 |
| Gly-Pro-Arg-Pro | 11.8 mM | 0.675 | 2.025 |
| Calcium | 162.2 nM | 0.375 | 1.125 |
| Thrombin (human) | 114 nM | 0.06 | 0.18 |
| Imidazole NaCl buffer | 50 mM | 1.44 | 4.32 |
| | 100 mM | | |
| Total | | 3 | 9 |

TABLE 7

| | | Volume in | Volume in |
|---|---|---|---|
| Component | Stock conc. | 10 μL (μL) | 30 μL (μL) |
| FX (human) | 4.8 μM | 0.083 | 0.25 |
| ATP | 20 mM | 0.85 | 2.55 |
| Magnesium | 500 mM | 0.083 | 0.25 |
| luciferin substrate | 10 mM | 1 | 3 |
| rLuciferase (Promega) | 12.5 mg/mL | 0.483 | 1.45 |
| Imidazole NaCl buffer | 50 mM | 1.5 | 4.5 |
| | 100 mM | | |

| | | |
|---|---|---|
| Total | 4 | 12 |

TABLE 8

| Component | Final concentration |
|---|---|
| FIXa (human) | 6.85 nM |
| Phospholipids | 10 μM |
| Gly-Pro-Arg-Pro | 0.8 mM |
| Calcium | 6 mM |
| Thrombin (human) | 0.7 nM |
| FX (human) | 40 nM |
| ATP | 1.7 mM |
| Magnesium | 4.2 mM |
| Luciferin substrate | 1 mM |
| rLuciferase (Promega) | 0.6 mg/mL |

### Procedure

The test is performed using a chemiluminescent reader at 37° C. The three mixtures are separately dispensed in a 384-wells microplate that was incubated at 37° C. Table 9 shows the division of only one well. During the test, the reactants are mixed and Relative Light Units (RLU) intensity is measured kinetically during 20 minutes. How to handle the final results is explained below.

TABLE 9

| | Volume in well 10 μL (μL) | Volume in well 30 μL (μL) |
|---|---|---|
| Reactant 1 | 3 | 9 |
| Reactant 2 | 3 | 9 |
| Reactant 3 | 4 | 12 |

### Calibration Curve

The chemiluminescence-based quantitative FVIII assay can be calibrated to analyze Factor VIII. The assay covers a dynamic range as shown in table 5. There are at least two methods to construct the calibration curve using the kinetic data. The first option is to set a time point after observing the raw data. A very suitable time point is considered when the curves of the individual calibrator samples form a plateau. The second option is determining the slope between approximately 0.5 to 3 minutes. The calibration curve is plotted log-log for both methods.
The calibration curve shown in the figures is obtained on a Flex3 Station (molecular devices, USA) in a final test volume of 10 pL. Based on this figure (raw data) the two options mentioned above were used to construct the final calibration curve.

An earlier version of this quantitative assay was mostly performed in 30 pL and with the QuantiLum luciferase (Promega). The latter is an import note considering this luciferase does not achieve the same intensity in the same environment as the Ultra-Glo luciferase which was used for the 10 pL structure. Therefore, the FIGS. 1-3 and 4-6 cannot be compared one on one. FIGS. 4 to 6 visualizes the results obtained with the 30 pL assay structure. Validation of this assay was performed with clinical samples of 31 hemophilia patients receiving FVIII replacement treatment. Twenty-two samples contained a recombinant PEGylated FVIII product, three of which were assigned lower than 1% FVIII and where recovered as lower than 1% FVIII. Ten samples contained a recombinant full-length FVIII product, one of which was assigned lower than 1% FVIII and was recovered as lower than 1% FVIII. These samples were measured both with a golden standard test as well as the luminescent-based FVIII assay. The golden standard assay utilized for the validation was a chromogenic FVIII assay containing bovine factors.

### Example 6

### Quantitative FIX Assay

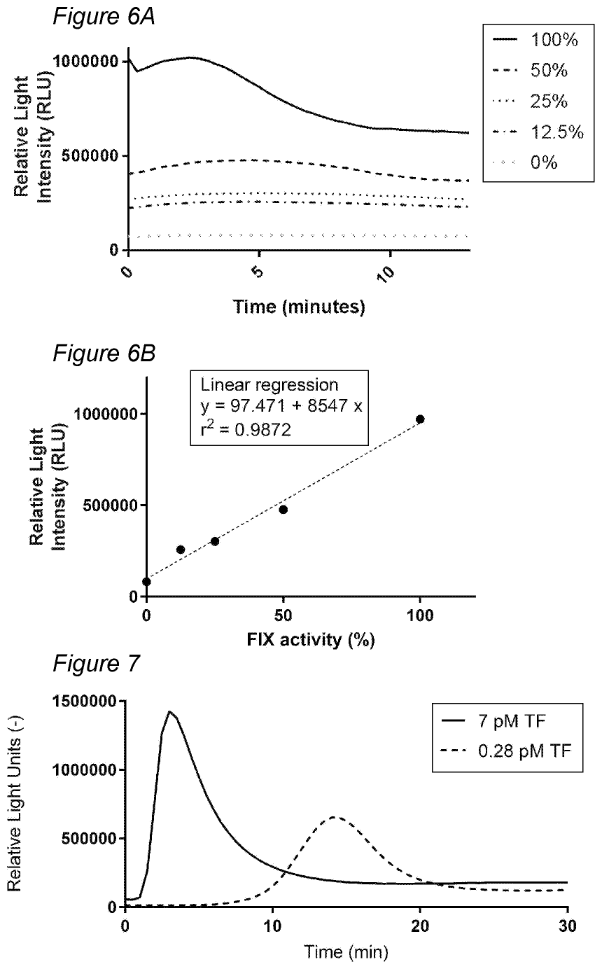

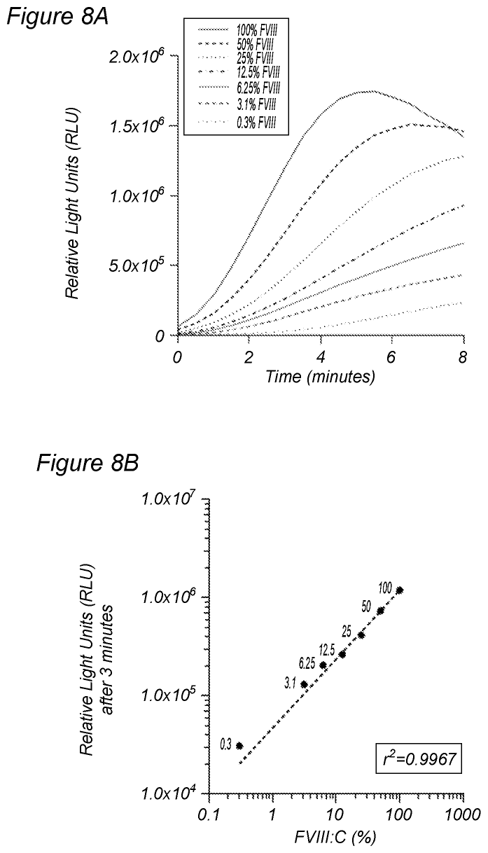

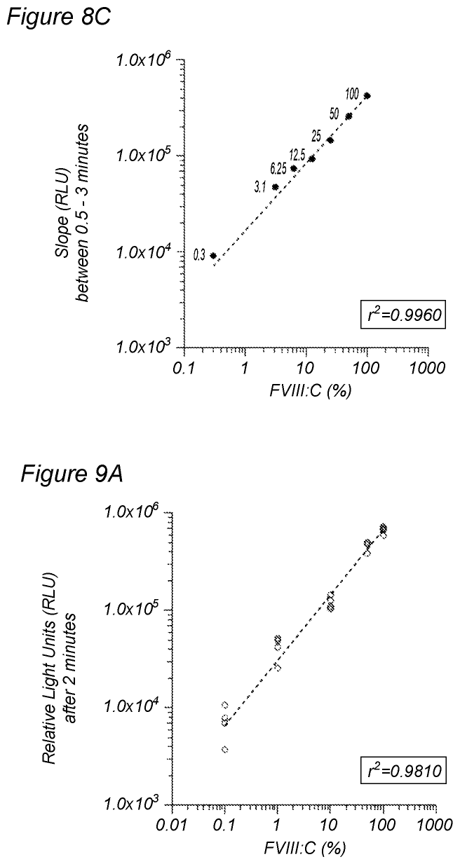

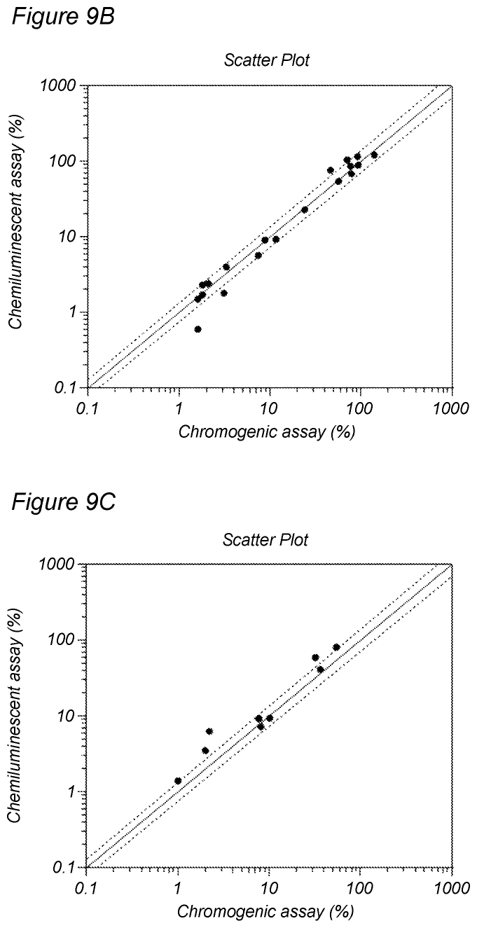

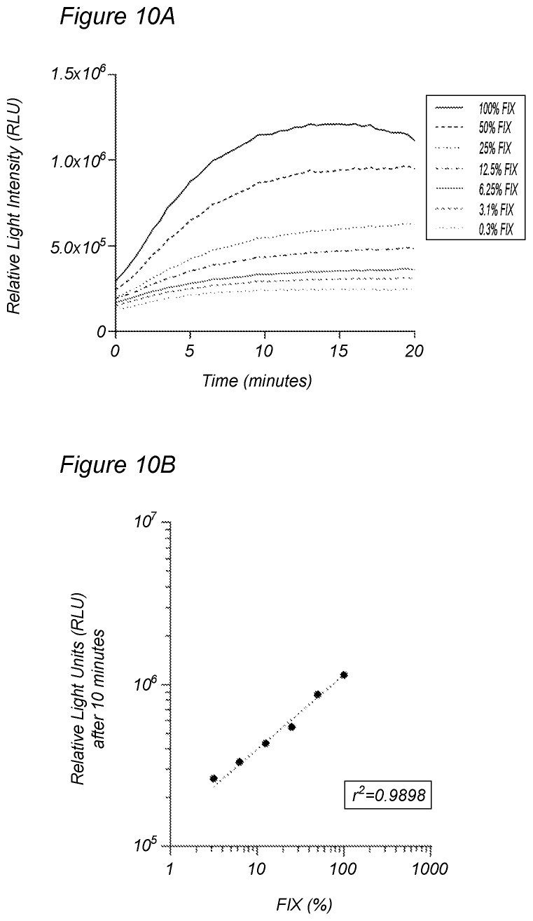

Using the same strategy as in Example 5 a luminescent FIX assay was designed. The assay design is comparable to the FVIII assay described above, but more specifically sensitized to FIX by providing an excess amount of FXIa instead of FIXa. FIX is a zymogen that can be cleaved by FXIa to produce FIXa. In the presence of Ca²⁺, membrane phospholipids, and FVIIIa, FIXa hydrolyses FX to form FXa. In the reaction mixture the factors of this cascade, except FIX which is to be assayed, were present in excess. The amount of FIX added to the reaction mixture thus leads to a correlated amount of FXa, which in turn leads to the detectable luminescence. Chromogenic analogues of such assays are commercially available (for example BIOPHEN Factor IX assay from Hyphen Biomed, Catalogue Ref. 221802). An important difference with this example is that the substrate according to the invention is used, together with Mg²⁺ and ATP and luciferin, instead of a chromogenic substrate.
Different amounts of FIX (using NPP or dilutions thereof) were added to a reaction mixture, after which luminescence was determined. FIG. 6A shows the RLU of FXa activity dependent on the FIX activity present in the sample, with added FIX expressed as NPP percentage. FIG. 6B shows the concomitant calibration line. The calibration has a linear regression of y=97471+8547x and r²=0.9872.

### Example 6.1

### Detailed FIX Assay

### Reagents

### Reagent 4 (R4):

Diluted plasma samples used for testing or for the calibration curve. Samples are diluted in buffer containing 50 mM imidazole and 100 mM NaCI buffer (pH 7.4).

### Reagent 5 (R5):

Recombinant human Factor VIII, human Factor Xla, human thrombin, synthetic phospholipids (28%), calcium and Gly-Pro-Arg-Pro as fibrin polymerization inhibitor . The individual components are stored at −80° C. in aqueous solution. The reagent mixture is prepared in buffer containing 50 mM imidazole and 100 mM NaCI buffer (pH 7.4).

### Reagent 6 (R6):

Human Factor X, ATP, magnesium, luminescent substrate specific for Factor Xa (in this example, MePEG2-IEGR was used) and the recombinant Luciferase Quantilum from Promega. The luciferase can be exchanged for the Ultra-Glo luciferase, both types can be applied. Magnesium is stored at 4-8° C. The individual components are stored at −80° C. in aqueous solution. The reagent mixture is prepared in buffer containing 50 mM imidazole and 100 mM NaCI (pH 7.4).

### Preparation Calibration Curve Samples

For the preparation of the calibration curve, the following diluted plasma samples are mixed as is shown in table 6 In the 384-well assay plate, 6 μL of the diluted sample or calibrator is used for the 30 μL test volume. Unknown specimens are prepared the same as the 100% samples.

TABLE 6

| | Stock solution | | | | | | | | |
|---|---|---|---|---|---|---|---|---|---|
| | Start | | Volume | | | Working solution | | | |
| | conc. | Volume | FVIIIdef | Total | | Volume | Volume | Total | Final |
| Cali- | (FVIII | start | plasma | volume | | stock | buffer | volume | conc. |
| brator | %) | (μL) | (μL) | (μL) | Dilution | (μL) | (μL)^{*} | (μL) | (FVIII %) |
| C1 | 100 | 40 | 0 | 40 | 7/60 | 7 | 53 | 60 | 100 |
| C2 | 100 | 20 | 20 | 40 | 7/60 | 7 | 53 | 60 | 50 |
| C3 | 50 | 20 | 20 | 40 | 7/60 | 7 | 53 | 60 | 25 |
| C4 | 25 | 20 | 20 | 40 | 7/60 | 7 | 53 | 60 | 12.5 |
| C5 | 12.5 | 20 | 20 | 40 | 7/60 | 7 | 53 | 60 | 6.25 |
| C6 | 6.25 | 20 | 20 | 40 | 7/60 | 7 | 53 | 60 | 3.125 |
| C7 | 3.125 | 10 | 90 | 100 | 7/60 | 7 | 53 | 60 | 0.3 |

^{*}buffer is 50 mM imidazole and 100 mM NaCl.

### Preparation Protein Reagents

Table 7and 8 show methods for preparing the reagents. These volumes represent 1 sample or 1 well in a 384 wells plate with a final test volume of 30 μL. The final concentration of each component in one sample is shown in table 9.

TABLE 7

| Component | Stock conc. | Volume in 30 μL (μL) |
|---|---|---|
| Recombinant FVIII | 12 IU/mL | 3 |
| FXIa (human) | 317 nM | 0.15 |
| Thrombin (human) | 114 nM | 0.26 |
| Phospholipids | 0.5 mM | 0.6 |
| Calcium | 162.2 mM | 1.1 |
| GPRP | 11.8 mM | 2 |
| Imidazole NaCl buffer | 50 mM 100 mM | 4.89 |

| | |
|---|---|
| Total | 12 |

TABLE 8

| Component | Stock conc. | Volume in 30 μL (μL) |
|---|---|---|
| FX (human) | 4.8 μM | 0.25 |
| ATP | 20 mM | 2.55 |
| Magnesium | 500 mM | 0.25 |
| luciferin substrate | 10 mM | 3 |
| rLuciferase (Promega) | 13.8 mg/mL | 1.5 |
| Imidazole NaCl buffer | 50 mM 100 mM | 4.45 |
| Total | | 12 |

TABLE 9
Final concentration of each component in one sample

| | Final |
|---|---|
| Component | concentration |
| Recombinant FVIII | 1.2 IU/mL |
| FX (human) | 40 nM |
| Luciferin substrate | 1 mM |
| FXIa (human) | 1.56 nM |
| Phospholipids | 10 μM |
| Calcium | 6 mM |
| Thrombin (human) | 1 nM |
| ATP | 1.7 mM |
| Magnesium | 4.2 mM |
| rLuciferase | 0.7 mg/mL |
| (Promega) | |

### Procedure

The test is performed using a chemiluminescent reader at 37° C. The four mixtures are separately dispensed in a 384-wells microplate that was incubated at 37° C. Table 10 shows the division of only one well. During the test, the Relative Light Units (RLU) intensity is measured kinetically.

TABLE 10

| | Volume in well 30 μL (μL) |
|---|---|
| Reagent 4 | 6 |
| Reagent 5 | 12 |
| Reagent 6 | 12 |

Mix and measure the RLU intensity for 20 minutes

### Calibration Curve

The FIX luminescent test can be calibrated for the assay of Factor IX or therapeutic concentrates. The plasma calibrators covering the suggested dynamic range are shown in table 6 and can be used to establish acalibration curve. First the raw data is visually assessed where after a suitable time point is selected to configure the calibration curves with. The calibration curve is plotted log-log.
The calibration curve shown in the figures is obtained on a Flex3 Station (molecular devices, USA). Based on this figure (raw data) the two options mentioned above were used to construct the final calibration curve.

### Example 7

### FXa Generation Assay

The substrate was also used in a global hemostasis assay, where FXa is generated in platelet poor plasma by activation of FX in the coagulation process upon initiation by tissue factor, phospholipids, and calcium. The FXa generation assay is initiated by addition of tissue factor and calcium to plasma, leading to FXa generation followed by thrombin generation and subsequent clot formation. FXa generation and its subsequent inhibition by physiological inhibitors leads to a bell-shaped curve (FIG. 7). Different doses of tissue factor were used: 7 μM or 0.28 μM. Measuring the FXa concentration in such a global design gives information about the capacity of the coagulation cascade at the level of factor Xa.

1. A compound of general formula (I-3) or (I-4), 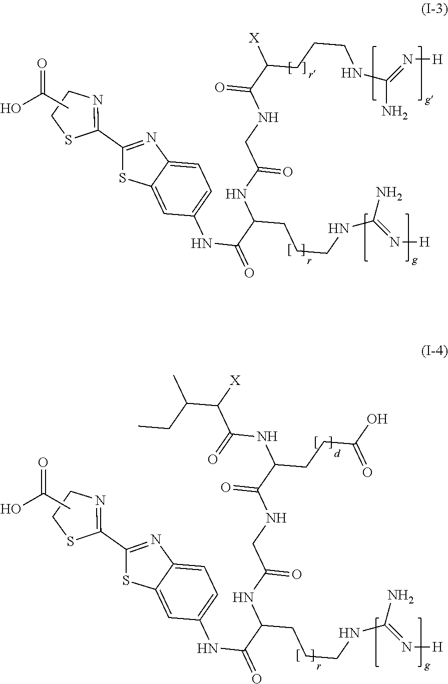
wherein
r is 0, 1, 2, or 3;
r′ is 0, 1, 2, or 3;
d is 0, 1, or 2;
g′ is 0 or1;
g′ is 0 or 1; and
X is a terminal moiety selected from NH₂, OH, O(C₁₋₆a lkyl), (OCH₂CH₂)₁₋₆OH, (OCH₂CH₂)₁₋₆(C₁₋₆alkyl), NHC(═O)(O)₀₋₁(C₁₋₆alkyl), NHC(═O)(O)₀₋₁(C₁₋₆alkylene)(OCH₂CH₂)₁₋₆OH, NHC(═O)(O)₀₋₁(C₁₋₆alkylene)(OCH₂CH₂)₁₋₆O(C₁₋₆alkyl), NHC(═O)(O)₀₋₁(C₁₋₆alkylene)O(C₁₋₆alkyl), NHC(═O)(O)₀₋₁(C₁₋₆alkylene)OH, and NP′ wherein P′ is an amine protecting group;
optionally wherein the aminoluciferin moiety is replaced by a different chemiluminescent amine;
or a physiologically acceptable salt thereof.

2. The compound according to claim 1, wherein d is 0 or 1-,

3. The compound according to claim 1, wherein r is 1 or 2, or wherein g is 1.

4. The compound according to claim 1, wherein the compound is of general formula (I-3s) or (I-4s), 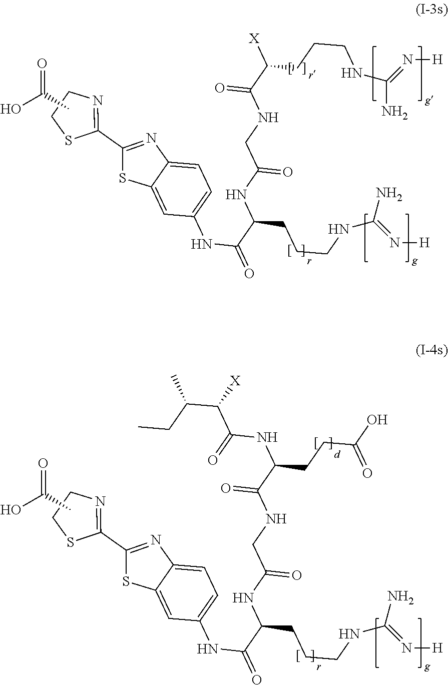
wherein
r is 0, 1, 2, or 3;
r′ is 0, 1, 2, or 3;
d is 0, 1, or 2;
g is 0 or 1;
g′ is 0 or 1.

5. The compound according to claim 1, wherein the compound is of general formula (II-3) or (II-4), 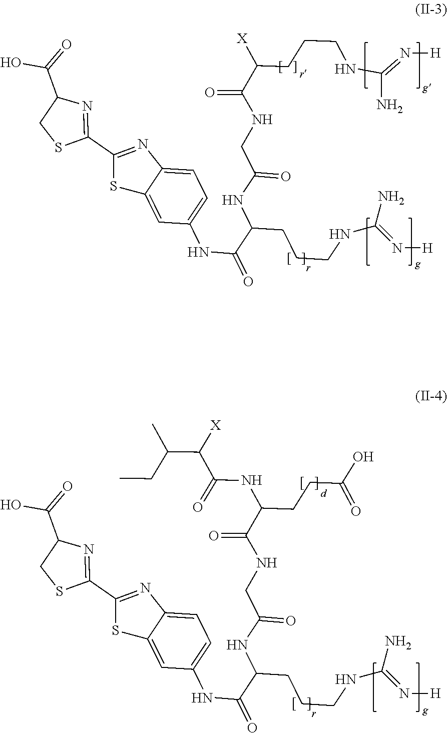
wherein
r is 0, 1, 2, or 3;
r′ is 0, 1, 2, or 3;
d is 0, 1, or 2;
g is 0 or 1;
g′ is 0 or 1; and
X is a terminal moiety selected from NH₂, OH, O(C1-6a1kyl), (OCH₂CH₂)1-6OH, (OCH2CH2)1 6O(C1 6alkyl), NHC(═O)(O)0-1(C1-6alkyl), NHC(═O)(O)0 1(C1-6alkylene)(OCH2CH2)1-6OH, NHC(═O)(O)0 1(C1 6alkylene)(OCH2CH2)1 6O(C1 6alkyl), NHC(═O)(O)0-1(C1 6alkylene)O(C1-6alkyl), NHC(═O)(O)0 1(C1-6alkylene)OH, and NP′ wherein P′ is an amine protecting group.

6. The compound according to claim 1, wherein the compound is of general formula (III-3) or (III-4), 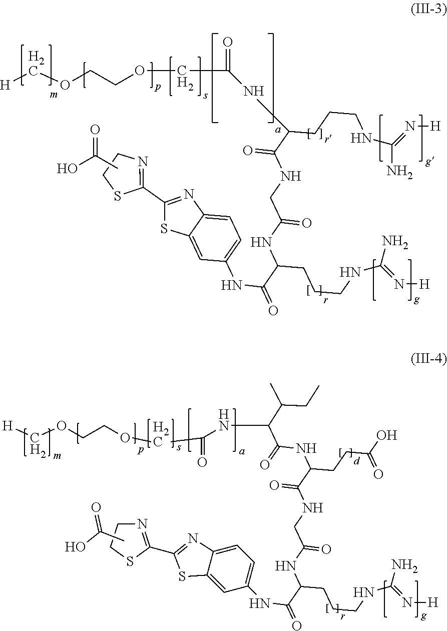
wherein
d is 0, 1, or 2;
r is 0, 1, 2, or 3;
r′ is 0, 1, 2, or 3;
g is 0 or1;
g′ is 0 or 1;
m is 0, 1, 2, 3, 4, 5, or 6;
p is 0, 1, 2, 3, 4, 5, or 6;
s is 0, 1, 2, 3, 4, 5, or 6; and
a is 0 or1.

7. The compound according to claim 6, wherein
d is 0 or 1, preferably 1; and/or
r is 1 or 2, preferably 1; and/or
r′ is 1 or 2, preferably 1; and/or
g is 0 or 1, preferably 1; and/or
g′ is 0 or 1, preferably 1; and/or
m is 0 or 1; and/or
p is 1, 2, or 3, preferably 2; and/or
s is 1 or 2, preferably 1; and/or
a is 1.

8. The compound according to claim 1, wherein the compound is selected from MePEG2-IEGR and MePEG2-IDGR

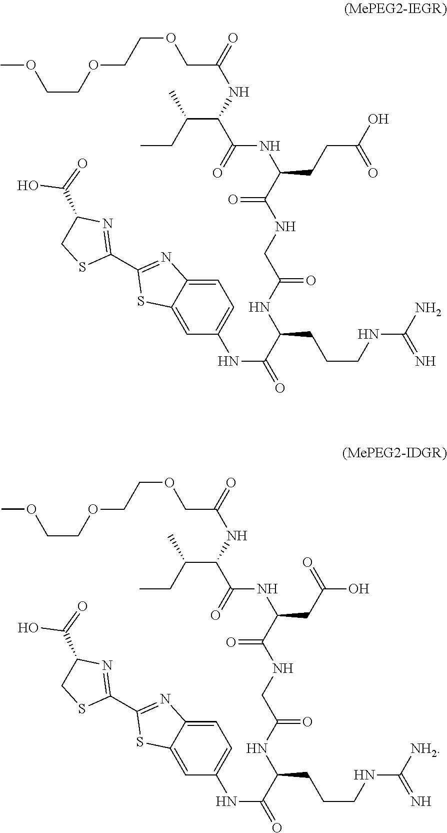 [SMILES: CC[C@H](C)[C@H](NC(=O)COCCOCCOC)C(=O)N[C@@H](CCC(=O)O)C(=O)NCC(=O)N[C@@H](CCCNC(=N)N)C(=O)Nc1ccc2nc(C3=N[C@@H](C(=O)O)CS3)sc2c1 ; CC[C@H](C)[C@H](NC(=O)COCCOCCOC)C(=O)N[C@@H](CC(=O)O)C(=O)NCC(=O)N[C@@H](CCCNC(=N)N)C(=O)Nc1ccc2nc(C3=N[C@@H](C(=O)O)CS3)sc2c1]

9. The compound according to claim 1, wherein P′ is selected from the group consisting of trityl, allyl, benzyl (Bn), benzoyl (Bz), 9-fluorenylmethyl oxycarbonyl (Fmoc), t-butyloxycarbonyl (Boc), benzyloxycarbonyl (Z), 2-trimethylsilylethyloxycarbonyl, tosyl (Ts), acetyl (Ac), trifluoroacetyl, phthalimide, benzylideneamine, and allyloxycarbonyl (Alloc), preferably from the group consisting of benzyl (Bn), 9-fluorenylmethyl oxycarbonyl (Fmoc), t-butyloxycarbonyl (Boc), benzyloxycarbonyl (Z), tosyl (Ts), and allyloxycarbonyl (Alloc), more preferably P′ is benzyloxycarbonyl.

10. The compound according to claim 1, wherein the compound is an acid addition salt optionally selected from a HCl salt, an acetic acid salt, a formic acid salt, a TFA salt, and a mesylic acid salt, preferably a HCl salt or a TFA salt, most preferably a TFA salt.

11.-12. (canceled)

13. Method for quantifying a coagulation factor in a sample, the method comprising the steps of:
a) contacting the sample with a composition comprising a compound as defined in claim 1 to release aminoluciferin;
b) contacting the aminoluciferin with luciferase; and
c) determining the relative light intensity generated by the luciferase.

14. The method according to claim 13, wherein the coagulation factor is selected from factor IX, factor IXa, factor VIII, factor VIIIa, factor VII, factor VIIa, factor XI, factor XIa, Factor XII, factor XIIa, factor X, factor Xa, prekallikrein, and kallikrein, optionally wherein during step a) the composition comprises at least one further coagulation factor selected from factor IX, factor IXa, factor VIII, factor VIIIa, factor VII, factor VIIa, factor XI, factor XIa, factor XII, factor XIIa, factor X, prekallikrein, and kallikrein.

15. Method for quantifying an anticoagulant in a sample, the method comprising the steps of:
a) contacting the sample with a composition comprising factor Xa and a compound as defined in claim 1 to release aminoluciferin;
b) contacting the aminoluciferin with luciferase; and
c) determining the relative light intensity generated by the luciferase.


SEQUENCE LISTING (4 sequences)

SEQ ID NO: 1 - protein, 4 aa, Artificial sequence
  Recognition sequence for FXa
IEGR

SEQ ID NO: 2 - protein, 4 aa, Artificial sequence
  Recognition sequence for FXa
IDGR

SEQ ID NO: 3 - protein, 4 aa, Artificial sequence
  Recognition sequence for FXa
IEGK

SEQ ID NO: 4 - protein, 4 aa, Artificial sequence
  Recognition sequence for FXa
IDGK